(19) United States
(12) Patent Application Publication
(10) Pub. No.: US 2012/0053350 A1
(43) Pub. Date: Mar. 1, 2012

(54) PREPARATION OF ALKYL ESTERS OF N-PROTECTED OXO-AZACYCLOALKYLCARBOXYLIC ACIDS

(76) Inventors: Ian Mangion, Cranford, NJ (US); Mark A. Huffman, Warren, NJ (US); Rebecca T. Ruck, Jersey City, NJ (US); Joseph Lynch, Plainfield, NJ (US); John Y.L. Chung, Edison, NJ (US); Benjamin Marcune, Raritan, NJ (US)

(21) Appl. No.: 13/266,871

(22) PCT Filed: Apr. 26, 2010
(86) PCT No.: PCT/US10/32380

§ 371 Date: Oct. 28, 2011

Related U.S. Application Data
(60) Provisional application No. 61/174,117, filed on Apr. 30, 2009.

(51) Int. Cl. C07D 471/08; C07D 401/12; C07D 211/60

(52) U.S. Cl. 546/121; 546/242; 546/208; 546/188

(57) ABSTRACT

A process for the preparation of alkyl esters of N-protected oxo-azacycloalkylcarboxylic acids of Formula III: comprises contacting a ketosulfoxonium ylide of Formula II: with an iridium catalyst to obtain Compound III, wherein P^{G1} is an amine protective group; k is 0, 1, or 2; and R^{U}, R¹, R², and R³ are defined herein. An embodiment of the process further com rises contacting a compound of Formula I: with a sulfoxonium halide of formula (R^{U})₃S(O)Z, wherein Z is halide, in the presence of a strong base to obtain Compound II. Additional embodiments add a series of process steps leading to the synthesis of 7-oxo-1,6-diazabicyclo[3.2.1]octanes suitable for use as β-lactamase inhibitors.

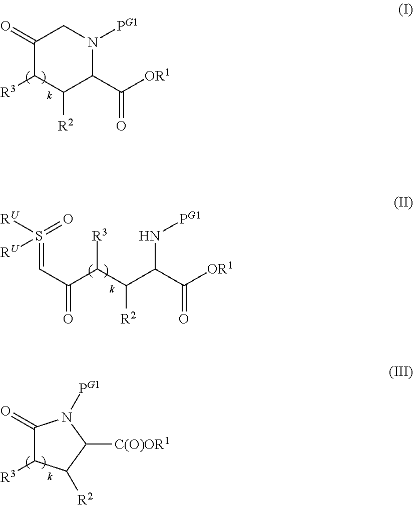

### CROSS-REFERENCE TO RELATED APPLICATIONS

This application claims the benefit of U.S. Provisional Application No. 61/174,117 (filed Apr. 30, 2009), the disclosure of which is hereby incorporated by reference in its entirety.

### FIELD OF THE INVENTION

The invention is directed to the preparation of alkyl esters of N-protected oxo-azacycloalkylcarboxylic acids. The esters are suitable for use as intermediates that lead via a series of additional process steps to the synthesis of 7-oxo-1,6-diazabicyclo[3.2.1]octane-2-carboxamides and -esters.

### BACKGROUND OF THE INVENTION

Certain 7-oxo-1,6-diazabicyclo[3.2.1]hexane-2-carboxamides are inhibitors of β-lactamase and, when used in conjunction with β-lactam antibiotics can be effective for the treatment of bacterial infections. WO 2009/091856 (corresponding to International Application No. PCT/US2009/031047, filed Jan. 15, 2009, and entitled “Beta-Lactamase Inhibitors”) discloses the synthesis of 7-oxo-1,6-diazabicyclo[3.2.1]hexane-2-carboxamides from a ketosulfoxonium ylide intermediate containing the amide side chain, wherein the ylide intermediate is cyclized to a 5-oxo-piperidine-2-carboxamide using an Ir, Rh, or Ru catalyst. The following exemplifies the chemistry disclosed in the document, wherein the desired diazabicyclohexane carboxamide compound is obtained in a subsequent convergent series of steps:

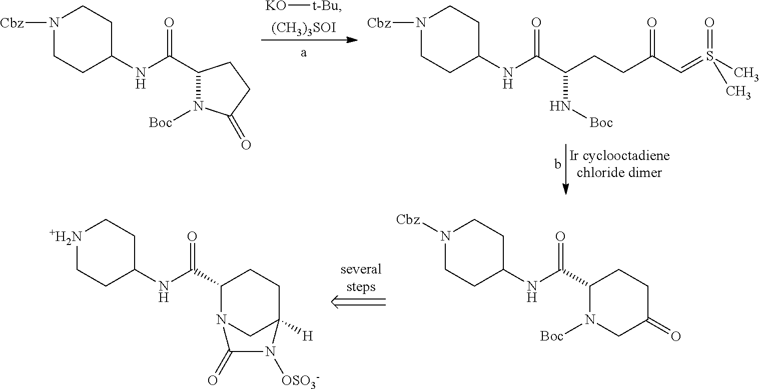

This process is an efficient means for synthesizing diazabicyclohexane carboxamides on a small or a large scale. However, the early introduction of the amide side chain effectively limits the process to the preparation of carboxamide final products. Furthermore, some amide side chains can be chemically unstable to reaction conditions required in one or more of the early synthetic steps of the disclosed process thereby limiting the application of the process. For example, compounds containing amide side chains having a functional group labile to basic conditions (e.g., ester, acetyloxy, or silyl ether) may not be suitable for use in the disclosed process due to the instability of the side chains to the potassium tert-butoxide chemistry employed in step a above. In addition, the disclosed process can be relatively expensive to operate in that the amide side chain in the final product is present in the first step; i.e., the disclosed process is a linearly sequential series of several steps leading to the final carboxamide product and there is generally a loss of material associated with each step (i.e., <100% yields in the steps due to the formation of by-products and/or losses associated with the recovery and isolation of the intermediate products). Accordingly, the amide material requirements can represent a significant cost, particularly when the side chain starting material is expensive to procure or prepare.

The following references are also of interest as background:

Baldwin et al., J. Chem. Soc., Chem. Commun. 1993, pp. 1434-1435 disclose the transformation of lactone-derived β-ketosulfoxonium ylides into β-oxonitrogen heterocycles in the presence of a rhodium catalyst. In particular, it was disclosed that the ring in 1-Boc-2-diphenylmethyloxycarbonyl-5-oxopyrrolidine was opened to the corresponding ylide which was then treated with Rh(II) trifluoroacetate to obtain 1-Boc-3-diphenylmethyloxycarbonyl-6-oxopiperidine.

US 2003/0199541 A1 discloses methods for preparing azabicyclic compounds which are useful as medicaments, in particular anti-bacterial agents.

WO 2008/039420 A2 discloses methods for preparing certain 7-oxo-2,6-diazabicyclo[3.2.0]heptane-2-carboxamides which are useful as β-lactamase inhibitors.

Mangion et al., Organic Letters 2009, vol. 11, pp. 3566-3569 disclose iridium-catalyzed X—H insertions (e.g., N—H insertions) of sulfoxonium ylides.

### SUMMARY OF THE INVENTION

The present invention includes a process for preparing a compound of Formula

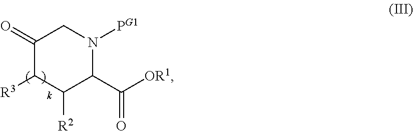

which comprises:

(B) contacting a ketosulfoxonium ylide of Formula II:

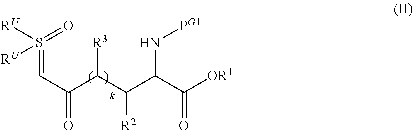

with an iridium catalyst to obtain Compound III; wherein:
P^{G1} is a first amine protective group which forms with the amino nitrogen to which it is attached a carbamate or a benzylamine;
each R^{U} is independently CH₃ or phenyl;
R¹ is C₁₋₆ alkyl or C₁₋₆ alkyl mono- or di-substituted with AryA, wherein each AryA is independently phenyl or napthyl and is optionally substituted with from 1 to 3 substituents each of which is independently halogen, C₁₋₆ alkyl, or O—C₁₋₆ alkyl;
k is an integer equal to 0, 1 or 2; and
R² and R³ are defined as follows:
(a) R² is H, C₁₋₆ alkyl, O—C₁₋₆ alkyl, O—Si(—C₁₋₆ alkyl)₃, or O—Si(—C₁₋₆ alkyl)(-phenyl)₂, and each R³ is H or C₁₋₆ alkyl; or
(b) alternatively and with the proviso that k is 1 or 2, R² and the R³ adjacent to R² together with the carbon atoms to which each is attached form C₅₋₇ cycloalkyl which is optionally substituted with from 1 to 3 substituents each of which is independently C₁₋₆ alkyl, O—C₁₋₆ alkyl, O—Si(—C₁₋₆ alkyl)₃, or O—Si(—C₁₋₆ alkyl)(phenyl)₂; and any other R³ is H or C₁₋₆ alkyl.

Compound III is useful as an intermediate that in combination with a series of additional steps (described below) results in a convergent synthesis of 7-oxo-1,6-diazabicyclo[3.2.1]octane-2-carboxamides and -2-carboxylic esters that can be used as β-lactamase inhibitors (BLIs). When a carboxamide BLI is desired, the use of the ester protecting group —C(O)OR¹ postpones the introduction of the amide side chain to a late stage of the convergent synthesis. The late introduction of the amide can provide an economic advantage with respect to a process such as the one described in the Background of the Invention in which the amide side chain—which can be expensive to procure or prepare—is introduced at or near the start of the synthesis in that the process of the invention can have a significantly smaller amide material requirement to prepare an equivalent amount of final product. The use of Compound III also provides for more flexibility in that it offers a more direct route to 7-oxo-1,6-diazabicyclo[3.2.1]octane-2-carboxylic esters suitable for use as BLIs. Furthermore, the use of Compound III permits the introduction of amide side chains that can be chemically unstable to reaction conditions required in early synthetic steps.

The Ir-catalyzed process of the invention can provide Compound III in significantly higher yields with lower catalyst loading in comparison to the Rh-catalyzed chemistry disclosed in Baldwin et al., J. Chem. Soc., Chem. Commun. 1993, pp. 1434-1435.

Other embodiments, aspects and features of the present invention are either further described in or will be apparent from the ensuing description, examples, and appended claims.

### DETAILED DESCRIPTION OF THE INVENTION

The present invention (alternatively referred to herein as “Process P”) includes a process for preparing an alkyl ester of Formula III which comprises Step B as set forth above in the Summary of the Invention. The amine protective group P^{G1}, in combination with the amino nitrogen to which it is attached, can be a carbamate (i.e., a protective group of formula

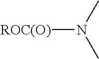

in which R is optionally substituted alkyl, allyl, optionally substituted benzyl, or the like) or a benzylamine (i.e., a protective group of formula

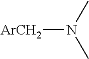

in which Ar is optionally substituted phenyl). Suitable carbamate and benzylamine protective groups and methods for their formation and cleavage are described in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991. In one embodiment, P^{G1} is (1) C(═O)—O—(CH₂)₀₋₁—CH═CH₂, (2) C(═O)—O—CH₂-AryB, wherein AryB is phenyl which is optionally substituted with from 1 to 3 substituents each of which is independently halo, —NO₂, —C₁₋₄ alkyl, or —O—C₁₋₄ alkyl, (3) C(═O)—O—C₁₋₄ alkyl, or (4) CH₂-AryC in which AryC is phenyl which is optionally substituted with from 1 to 3 substituents each of which is independently halo, —NO₂, —C₁₋₄ alkyl, or —O—C₁₋₄ alkyl. In another embodiment, P^{G1} is t-butyloxycarbonyl (Boc), allyloxycarbonyl (Allot), benzyloxycarbonyl (Cbz), p-methoxybenzyloxycarbonyl, p-nitrobenzyloxycarbonyl, p-bromobenzyloxycarbonyl, p-chlorobenzyloxycarbonyl, 2,4-dichlorobenzyloxycarbonyl, or benzyl. In still another embodiment, P^{G1} is Boc.

Other embodiments of Compound III and Step B include the following:

(1a) both R^{U} are CH₃;
(1b) both R^{U} are phenyl;
(1c) one R^{U} is CH₃, and the other R^{U} is phenyl;
(2a) R¹ is C₁₋₄ alkyl or C₁₋₄ alkyl mono- or di-substituted with AryA, wherein each AryA is independently phenyl or napthyl and is optionally substituted with from 1 or 2 substituents each of which is independently C₁₋₄ alkyl, or O—C₁₋₄ alkyl;
(2b) R¹ is C₁₋₄ alkyl, benzyl or diphenylmethyl;
(2c) R¹ is C₁₋₄ alkyl;
(2d) R¹ is branched C₃₋₆ alkyl;
(2e) R¹ is isopropyl, t-butyl, sec-butyl, isobutyl, isopentyl, or neopentyl;
(2f) R¹ is t-butyl;
(3a) k is 0 or 1;
(3b) k is 0;
(3c) k is 1;
(4a) R² is H, C₁₋₄ alkyl, O—C₁₋₄ alkyl, O—Si(—C₁₋₄ alkyl)₃, or O—Si(—C₁₋₄ alkyl)(phenyl)₂, and each R³ is H or C₁₋₄ alkyl;
(4b) R² is H, CH₃, OCH₃, O-trimethylsilyl (TMS), O-t-butyldiphenylsilyl (TBDPS), O-t-butyldimethylsilyl (TBS), or O-triisopropylsilyl (TIPS), and each R³ is H or CH₃;
(4c) R² is H or CH₃, and each R³ is H or CH₃;
(4d) R² is H, and each R³ is H;
(4e) with the proviso that k is 1 or 2, R² and the R³ adjacent to R² together with the carbon atoms to which each is attached form C₅₋₆ cycloalkyl; and any other R³ is H.

One or more of these embodiments (1) to (4) can be combined with each other and/or with the embodiments described above for P^{G1}, wherein each such combination is a separate embodiment of Compound III and Step B.

Step B involves the intramolecular insertion of NH using a ketosulfoxonium ylide to form a cyclic product. The ylide chemistry employed in Step A provides a safety benefit with respect to alternative methods that employ diazomethane (an explosion hazard) to generate a diazoketone which can then be used in a cyclization. Step B can also provide a high yield; i.e., yields of 60% or higher.

Step B is conducted in an organic solvent. Suitable solvents include toluene, dichloromethane, DCE, DMF, THF, chlorobenzene, 1,2-dichlorobenzene, cyclopentylmethyl ether, acetonitrile, IPAc, nitromethane, trifluoromethylbenzene, methyl ethyl ketone, DME, and 2-MeTHF. A preferred solvent is DCE.

The cyclization in Step B is conducted in the presence of an Ir catalyst. Suitable catalysts include [Ir(COD)Cl]₂, (COD)₂IrBF₄, IrCl(CO)(PPh₃)₂, IrCl(CO)₃, Ir(COD)(acac), Ir(CO)₂(acac), (methylcyclopentadienyl)(COD)Ir, ((cyclohexyl)₃P)₃(COD)Ir(pyridine), and Ir(COD)₂BARF. A class of suitable catalysts consists of ([Ir(COD)Cl]₂), Ir(COD)₂BF₄, and Ir(COD)₂BARF. A preferred catalyst is [Ir(COD)Cl]₂. The catalyst is typically employed in an amount in a range of from about 0.25 to 5 mole percent based on the amount of Compound II, and is more typically employed in an amount in a range of from about 0.5 to about 2 mole percent.

The reaction in Step B can suitably be conducted at a temperature in a range of from about 50° C. to about 130° C. and is typically conducted at a temperature in a range of from about 70° C. to about 110° C.

An embodiment of Process P comprises Step B as just described above and further comprises:

(A) contacting a compound of Formula I:

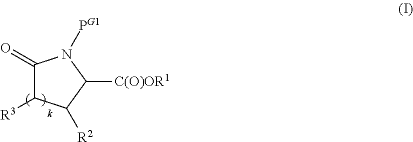

with a sulfoxonium compound of formula (R^{U})₃S(O)Z, wherein at least one R^{U} is CH₃ and Z is halide (e.g., chloride, bromide or iodide) or tetrafluoroborate, in the presence of strong base to obtain Compound II.

Step A is conducted in an organic solvent. Suitable solvents include toluene, dichloromethane, DCE, DMF, THF, chlorobenzene, 1,2-dichlorobenzene, cyclopentylmethyl ether, acetonitrile, IPAc, nitromethane, trifluoromethylbenzene, methyl ethyl ketone, DME, and 2-MeTHF. Preferred solvents are DCE, DMF and toluene.

Suitable sulfoxonium compounds in Step A include trimethylsulfoxonium chloride, trimethylsulfoxonium bromide, trimethylsulfoxonium iodide, diphenylmethylsulfoxonium chloride, and diphenylmethylsulfoxonium tetrafluoroborate. A class of suitable halides consists of trimethylsulfoxonium chloride, trimethylsulfoxonium bromide, and trimethylsulfoxonium iodide. Preferred halides include trimethylsulfoxonium chloride and trimethylsulfoxonium iodide. The sulfoxonium halide is typically employed in an amount in a range of from about 1.0 to about 2.5 equivalents per equivalent of Compound I, and is more typically employed in an amount in a range of from about 1.2 to about 1.6 equivalents.

The reaction in Step A can suitably be conducted at a temperature in a range of from about −10° C. to about 40° C. and is typically conducted at a temperature in a range of from about 0° C. to about 25° C.

An embodiment of Process P comprises Step B as just described above or Steps A and B as just described, and further comprises:

(C) treating Compound III with a reducing agent to obtain a compound of Formula IV:

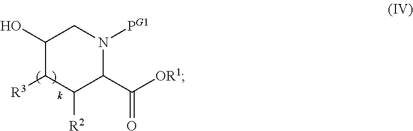

and

(D) contacting Compound IV with a sulfonyl halide of formula IV-Su:

R⁴—SO₂W (IV-Su)

in the presence of a tertiary amine base to obtain a compound of Formula V:

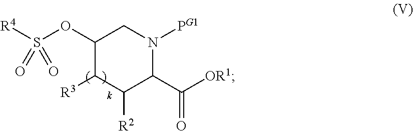

wherein W is halogen; and R⁴ is:
(1) phenyl optionally substituted with from 1 to 3 substituents each of which is independently C₁₋₄ alkyl, C₁₋₄ haloalkyl, O—C₁₋₄ alkyl, O—C₁₋₄ haloalkyl, Cl, Br, F, or NO₂;
(2) C₁₋₄ alkyl; or
(3) C₁₋₄ haloalkyl.

Step C is conducted in an organic solvent. Suitable solvents include toluene, dichloromethane, THF, isopropyl alcohol, and acetonitrile. Preferred solvents are toluene and THF.

Suitable reducing agents in Step C include LiBH₄, NaBH₄, KBH₄, (Me₄N)BH₄, LiAlH(O-t-Bu)₃, LiBH(OEt)₃, and Al(O-i-Pr)₃/IPA. A class of suitable reducing agents consists of LiBH₄, NaBH₄, and KBH₄. Preferred reducing agents include LiBH₄ and NaBH₄. The reducing agent is typically employed in an amount in a range of from about 1 to about 2 equivalents per equivalent of Compound III, and is more typically employed in an amount in a range of from about 1 to about 1.3 equivalents.

The reaction in Step C can suitably be conducted at a temperature in a range of from about −20° C. to about 40° C. and is typically conducted at a temperature in a range of from about −15° C. to about 0° C.

Step D is conducted in an organic solvent. Suitable solvents include dichloromethane, THF, ethyl acetate, and MTBE. A preferred solvent is dichloromethane.

Exemplary sulfonyl halides suitable for use in Step D include methanesulfonyl chloride, chloromethanesulfonyl chloride, dichloromethanesulfonyl chloride, benzenesulfonyl chloride, p-trifluoromethylbenzenesulfonyl chloride, p-toluenesulfonyl chloride, p-bromobenzenesulfonyl chloride, p-fluorobenzenesulfonyl chloride, p-methoxybenzenesulfonyl chloride, and 2,4-dichlorobenzenesulfonyl chloride. A class of suitable sulfonyl halides consists of chloromethanesulfonyl chloride, p-trifluoromethylbenzenesulfonyl chloride, p-bromobenzenesulfonyl chloride, and 2,4-dichlorobenzenesulfonyl chloride. Another class of suitable sulfonyl halides consists of chloromethanesulfonyl chloride, p-trifluoromethylbenzenesulfonyl chloride and p-bromobenzenesulfonyl chloride. A preferred sulfonyl halide is p-trifluoromethylbenzenesulfonyl chloride. Another preferred sulfonyl halide is 2,4-dichlorobenzenesulfonyl chloride. The sulfonyl halide is typically employed in an amount in a range of from about 1 to about 2 equivalents per equivalent of Compound IV, and is more typically employed in an amount in a range of from about 1 to about 1.5 equivalents (e.g., about 1.3 equivalents).

The tertiary amine base in Step D is suitably a tri-C₁₋₄ alkylamine. A class of suitable amines consists of TEA, DIPEA, and diethylisopropylamine. TEA is a preferred base. The base is typically employed in an amount in a range of from about 1 to about 3 equivalents per equivalent of Compound IV, and is more typically employed in an amount in a range of from about 1.1 to about 2 equivalents (e.g., about 1.8 equivalents).

The reaction in Step D can suitably be conducted at a temperature in a range of from about 0° C. to about 40° C. and is typically conducted at a temperature in a range of from about 10° C. to about 25° C.

Another embodiment of Process P comprises Steps B to D as described above or Steps A to D as described above, and further comprises:

(E) treating Compound V with a P^{G1}-cleaving agent to obtain a compound of Formula VI:

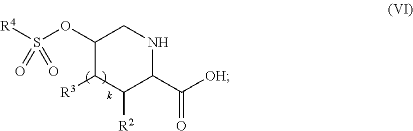

and

(F) treating Compound VI with a P^{G2}-producing agent to obtain a compound of Formula VII:

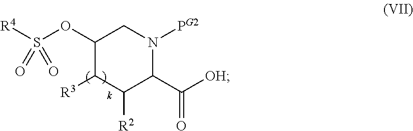

wherein:
P^{G2} is amine protective group which forms with the amino nitrogen to which it is attached an alkyl carbamate;

Step E is conducted in an organic solvent. Suitable solvents include DCE, toluene, DMF, acetonitrile and dichloromethane. Preferred solvents are acetonitrile and dichloromethane.

The choice of P^{G1}-cleaving agent depends upon the nature of the P^{G1} group. In most cases the group can be cleaved by treatment with acid, such as a mineral acid, a Lewis acid, or an organic acid. Suitable mineral acids include hydrogen halides (HCl, HBr, and HF, as a gas or in aqueous solution), sulfuric acid, and nitric acid. Suitable organic acids include carboxylic acids, alkylsulfonic acids and arylsulfonic acids. Exemplary organic acids include trifluoroacetic acid (TFA), toluenesulfonic acid, benzenesulfonic acid, methanesulfonic acid, and trifluoromethanesulfonic acid. Suitable Lewis acids include BF₃.Et₂O, SnCl₄, ZnBr₂, Me₃Sil, Me₃SiCl, Me₃SiOTf, and AlCl₃. Cleavage conditions (e.g., temperature, choice and concentration of acid) can vary from mild to harsh depending upon the lability of the amino protective group. While treatment with an acid is typically effective, other cleaving agents can be employed. Certain P^{G1} groups such as Cbz or Alloc, for example, can be efficiently cleaved via hydrogenolysis (e.g., hydrogenation with a Pd catalyst). Further description of cleaving agents and deprotection treatments suitable for use in Step E can be found in Protective Groups in Organic Chemistry, ed. J. F. W. McOmie, Plenum Press, 1973 and in T. W. Greene & P. G. M. Wuts, Protective Groups in Organic Synthesis, John Wiley & Sons, 3^{rd} edition, 1999, and 2^{nd} edition, 1991.

The cleaving agent in Step E is typically employed in an amount in a range of from about 2.0 to about 15.0 equivalents per equivalent of Compound V, and is more typically employed in an amount in a range of from about 2.5 to about 5.0 equivalents.

The reaction in Step E can suitably be conducted at a temperature in a range of from about −10° C. to about 60° C. and is typically conducted at a temperature in a range of from about 0° C. to about 40° C.

Step F is conducted in an organic solvent. Suitable solvents include dichloromethane, acetonitrile, THF, and DCE. Preferred solvents are dichloromethane and acetonitrile.

P^{G2} is an acid-labile amine protective group. The reference to the P^{G2} group as being “acid labile” means it can be removed by treatment with an acid to provide the free amine. Suitable acids are the same as those described above with respect to the cleavage of P^{G1} in Step E. P^{G2}, in combination with the amino nitrogen to which it is attached, is suitably an alkyl carbamate. In one embodiment, P^{G2} is C(═O)—O—C₁₋₄ alkyl. A preferred P^{G2} group is t-butyloxycarbonyl (Boc).

P^{G2}-producing agents corresponding to the P^{G2} groups set forth above and suitable for use in Step F are, for example, C₁₋₄ alkyl-O—C(═O)—Y, wherein Y is halide (e.g., chloride), and [C₁₋₄ alkyl-O—C(═O)]₂O. Other suitable P^{G2}-producing agents are known. For example, di-t-butyl carbonate and t-butylchloroformate are effective Boc-producing agents, but Boc can also be produced using Boc-ON or Boc-OSN.

The P^{G2}-producing agent is typically employed in Step F in an amount in a range of from about 1.0 to about 2.0 equivalents per equivalent of Compound VI, and is more typically employed in an amount in a range of from about 1.0 to about 1.3 equivalents.

The reaction in Step F can suitably be conducted at a temperature in a range of from about 0° C. to about 40° C. and is typically conducted at a temperature in a range of from about 10° C. to about 25° C.

Another embodiment of Process P comprises Steps B to F as described above or Steps A to F as described above, and further comprises:

(G) contacting Compound VII with an azacycloalkylamine of formula VII-Am:

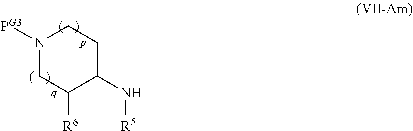

in the presence of a coupling agent to obtain an amide of Formula VIII:

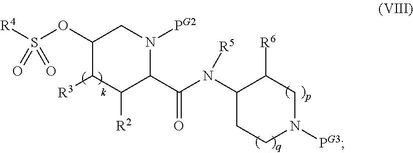

wherein:
P^{G3} is a third amine protective group selected from the group consisting of (i) carbamates other than alkyl carbamates and (ii) benzylamines;
R⁵ is H or C₁₋₃ alkyl;
R⁶ is H, Cl, Br, F, C₁₋₃ alkyl, O—C₁₋₃ alkyl, or N(—C₁₋₃ alkyl)₂;
p is zero, 1 or 2;
q is zero, 1, or 2; and
p+q=zero, 1, 2, or 3.

P^{G3} is an amine protective group which is not acid-labile under conditions in which the P^{G2} group is acid labile. In other words, P^{G3} is a group which is not cleaved under acidic conditions suitable for the removal of P^{G2}. P^{G3}, in combination with the amino nitrogen to which it is attached, is suitably an aryl carbamate, vinyl carbamate, allyl carbamate, or a benzylamine. In one embodiment, P^{G3} is (1) C(═O)—O—(CH₂)₀₋₁—CH═CH₂, (2) C(═O)—O—CH₂-AryD wherein AryD is phenyl which is optionally substituted with from 1 to 3 substituents each of which is independently halo, —NO₂, —C₁₋₄ alkyl, or alkyl, or (3) CH₂-AryE wherein AryE is phenyl which is optionally substituted with from 1 to 3 substituents each of which is independently halo, —NO₂, —C₁₋₄ alkyl, or —O—C₁₋₄ alkyl. Suitable P^{G3} groups include Cbz, Alloc, para-methoxy benzyl, and benzyl. A preferred P^{G3} is Cbz.

P^{G3}-producing agents corresponding to the P^{G3} groups set forth above and suitable for use in Step F are, for example, (1) CH═CH₂—(CH₂)₀₋₁—O—C(═O)—Y or [CH═CH₂—CH₂—O—C(═O)]₂O, (2) AryD-O—C(═O)—Y or [AryD-O—C(═O)]₂O, or (3) AryE-CH₂—Y.

Other embodiments of Step G include the following features of amine VII-Am:

### (5a) R⁵ is H or CH₃;

### (5b) R⁵ is H;

(6a) R⁶ is H or C₁₋₃ alkyl;

### (6b) R⁶ is H or CH₃;

### (6e) R⁶ is H;

(7a) p is 1 and q is 1 (i.e., the compound is a 4-piperidinylamine);
(7b) p is 1 and q is 0 (i.e., the compound is a 3-pyrrolidinylamine).

One or more of these embodiments (5) to (7) can be combined with each other and/or with the embodiments described above for P^{G3}, wherein each such combination is a separate embodiment of the amine compound employed in Step G.

Amines of Formula VII-Am can be prepared, for example, by reductive amination of the corresponding ketone or by hydride reduction of the corresponding imine. Further description of methods suitable for the preparation of amines of Formula VII-Am can be found in Richard Larock, Comprehensive Organic Transformations, 2^{nd} edition, Wiley-VCH Publishers Inc, 1999, pp 753-879.

Step G involves the coupling of azacycloalkylamine VII-Am with carboxylic acid VII to obtain the amide VIII. Suitable coupling agents in Step G include DCC, EDC, HATU, TBTU, PyBOP, DPPA, and BOP-Cl. Preferred agents are DCC and EDC. The coupling agent is typically employed in an amount in a range of from about 1.0 to about 1.5 equivalents per equivalent of Compound VII, and is more typically employed in an amount in a range of from about 1.0 to about 1.2 equivalents. Coupling additives such as HOBt, HOAt, or HOPO can also be employed. The coupling reaction is suitably conducted in the presence of a base such as a trialkylamine (e.g., TEA or DIPEA).

Step G is conducted in an organic solvent. Suitable solvents include dichloromethane, DCE, THF, DMF, NMP, 1,4-dioxane, dimethylacetamide, and acetonitrile. Preferred solvents are dichloromethane and DMF.

The coupling in Step G can suitably be conducted at a temperature in a range of from about −10° C. to about 40° C. and is typically conducted at a temperature in a range of from about 0° C. to about 25° C.

Another embodiment of Process P comprises Steps B to G as described above or Steps A to G as described above, and further comprises:

(H) contacting Compound VIII with N-Boc-O-benzylhydroxylamine in the presence of a base to obtain a compound of Formula IX:

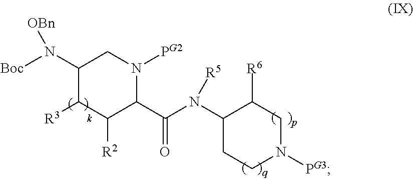

and

(I) treating Compound IX with an acid to obtain a compound of Formula X:

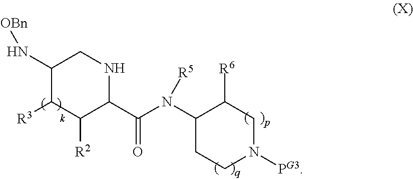

Step H is conducted in an organic solvent. Suitable solvents include DMAC, DMF, NMP, THF and DME. A preferred solvent is NMP.

Suitable bases in Step H include Li t-butoxide, Na t-butoxide, K t-butoxide, cesium carbonate, KHMDS, and NaHMDS. A class of suitable bases consists of Li t-butoxide, Na t-butoxide, K t-butoxide and cesium carbonate. Preferred bases are K t-butoxide and cesium carbonate. The base is typically employed in an amount in a range of from about 1 to about 2 equivalents per equivalent of Compound VIII, and is more typically employed in an amount in a range of from about 1 to about 1.5 equivalents (e.g., about 1.3 equivalents).

The N-Boc-O-benzylhydroxylamine is typically employed in Step H in an amount in a range of from about 1 to about 2 equivalents per equivalent of Compound VIII, and is more typically employed in an amount in a range of from about 1 to about 1.5 equivalents (e.g., about 1.3 equivalents).

The reaction in Step H can suitably be conducted at a temperature in a range of from about 30° C. to about 60° C. and is typically conducted at a temperature in a range of from about 35° C. to about 45° C.

Step I is conducted in an organic solvent. Suitable solvents include DCM and acetonitrile.

Suitable acids in Step I include sulfonic acids. Suitable acids in Step I include methanesulfonic acid, trifluoromethane sulfonic acid, chloromethanesulfonic acid, benzenesulfonic acid, p-toluenesulfonic acid, p-bromobenzenesulfonic acid, p-methoxybenzenesulfonic acid, and p-trifluoromethylbenzenesulfonic acid. A class of suitable acids consists of p-toluenesulfonic acid and methanesulfonic acid. A preferred acid is methanesulfonic acid. The acid is typically employed in an amount in a range of from about 1 to about 6 equivalents per equivalent of Compound IX, and is more typically employed in an amount in a range of from about 3 to about 5 equivalents.

The reaction in Step I can suitably be conducted at a temperature in a range of from about 25° C. to about 60° C. and is typically conducted at a temperature in a range of from about 30° C. to about 40° C.

Another embodiment of Process P comprises Steps B to I as described above or Steps A to I as described above, and further comprises:

(J) contacting Compound X with phosgene, diphosgene or triphosgene in the presence of a tertiary amine, and then adding an aqueous solution of acid to obtain a compound of Formula XI:

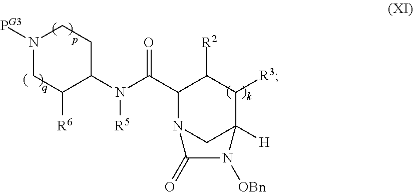

and

(K) contacting Compound XI with a source of hydrogen in the presence of a hydrogenolysis catalyst and in the presence of a Boc-producing agent to obtain a compound of Formula XII:

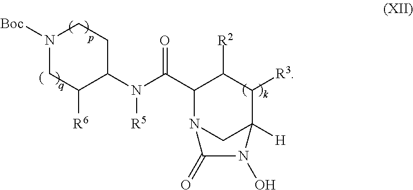

Step J is conducted in an organic solvent. Suitable solvents include DCM and acetonitrile. A preferred solvent is DCM.

Suitable acids in Step J include hydrochloric acid, sulfuric acid, trifluoroacetic acid, and phosphoric acid. A preferred acid is phosphoric acid. The acid is typically employed in an amount in a range of from about 1 to about 6 equivalents per equivalent of Compound X, and is more typically employed in an amount in a range of from about 3 to about 5 equivalents (e.g., about 3.2 equivalents).

The tertiary amine in Step J is suitably a tri-C₁₋₄ alkylamine. A class of suitable amines consists of TEA, DIPEA, and diethylisopropylamine. DIPEA is a preferred amine. The amine is typically employed in an amount in a range of from about 1 to about 6 equivalents per equivalent of Compound X, and is more typically employed in an amount in a range of from about 3 to about 5 equivalents (e.g., about 3.2 equivalents).

The triphosgene, diphosgene, or phosgene is typically employed in Step 3 in an amount in a range of from about 0.5 to 1 equivalents per equivalent of Compound X, and is more typically employed in an amount in a range of from about 0.7 to about 1 equivalent (e.g., about 0.8 equivalent). Triphosgene is preferred over diphosgene and phosgene.

The contacting of Compound X with triphosgene, diphosgene, or phosgene in Step 3 can suitably be conducted at a temperature in a range of from about −15° C. to about 40° C. and is typically conducted at a temperature in a range of from about −5° C. to about 25° C. The subsequent addition and reaction with the acid can suitably be conducted at a temperature in a range of from about 0° C. to about 25° C.

Step K is conducted in an organic solvent. Suitable solvents include ethyl acetate, DMAC, t-butanol, and THF. A preferred solvent is THF.

Suitable Boc-producing agents in Step K include di-t-butyl carbonate, t-butylchloroformate, Boc-ON and Boc-OSN. A preferred agent is di-t-butyl carbonate. The agent is typically employed in an amount in a range of from about 0.9 to about 3 equivalents per equivalent of Compound XI, and is more typically employed in an amount in a range of from about 0.9 to 1.5 equivalents (e.g., from about 0.95 to about 1.1 equivalents).

The P^{G3} group is removed in Step K by hydrogenolysis. The source of hydrogen in Step K is typically hydrogen gas, optionally in admixture with a carrier gas that is chemically inert under the reaction conditions employed in Step K (e.g., nitrogen or a noble gas such as helium or argon). The pressure is not a critical aspect in Step K, although atmospheric and superatmospheric pressures tend to be expedient. The pressure typically is at least about 2 psig (about 115 kPa). The hydrogen source can alternatively be a hydrogen-transfer molecule such as ammonium formate, cyclohexene, or cyclohexadiene.

The uptake of hydrogen is not a critical process parameter, although at least a stoichiometric amount of hydrogen gas or other hydrogen source is typically employed.

The hydrogenolysis catalyst comprises a supported or unsupported Group 8 metal or a supported or unsupported compound, salt or complex of a Group 8 metal. The catalyst typically employed in Step K is supported or unsupported Pd metal or a supported or unsupported Pd compound, salt or complex. Suitable catalyst supports include carbon, silica, alumina, silicon carbide, aluminum fluoride, and calcium fluoride. A class of suitable catalysts consists of Pd black (i.e., fine metallic palladium particles), Pd(OH)₂, and Pd/C (i.e., palladium on a carbon support). Pd/C is a preferred hydrogenolysis catalyst. The catalyst is typically employed in an amount in a range of from about 5 to about 20 wt. % relative to the amount of Compound XI, and is more typically employed in an amount in a range of from about 5 to about 15 wt. % (e.g., about 10 wt. %).

The reaction in Step K can suitably be conducted at a temperature in a range of from about 10° C. to about 50° C. and is typically conducted at a temperature in a range of from about 15° C. to about 30° C.

Another embodiment of Process P comprises Steps B to K as described above or Steps A to K as described above, and further comprises:

(L) contacting Compound XII with a sulfating agent in the presence of an organic base to obtain a compound of Formula XIII:

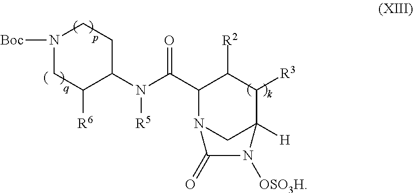

The sulfating agent in Step L is suitably a complex of sulfur trioxide and an amine, wherein the amine is suitably a tertiary amine including, for example, acyclic amines (e.g., trimethylamine, TEA, dimethylphenylamine and dimethylbenzylamine), cyclic amines (e.g., 1-methylpyrrolidine and 1-methylpiperidine) and aromatic amines having one or more N atoms as part of the aromatic ring (e.g., 1-methylimidazole, pyridine, and pyrimidine). Halosulfonic acids (e.g., chlorosulfonic acid) and tertiary amide complexes of SO₃ (e.g., DMF-SO₃) are also suitable sulfating agents. A class of suitable sulfating agents consists of complexes of each of the following amines with sulfur trioxide: pyridine, trimethylamine, and triethylamine. Another class of suitable sulfating agents consists of pyridine-SO₃ complex, DMF-SO₃ complex and chlorosulfonic acid. The sulfating reagent is typically employed in an amount in a range of from about 1.5 to about 7.0 equivalents per equivalent of Compound XII, and is more typically employed in an amount in a range of from about 3.0 to about 4.5 equivalents.

The organic base is suitably a tertiary amine such as 2-picoline, 2,6-lutidine, an individual trimethylpyridine, or a mixture of two or more trimethylpyridines. A class of suitable bases consists of 2-picoline, 2,6-lutidine and 2,4,6-trimethylpyridine. In a preferred embodiment, the base is 2-picoline. The base is typically employed in an amount in a range of from about 1 to about 3 equivalents per equivalent of Compound XII, and is more typically employed in an amount in a range of from about 1.7 to about 2.2 equivalents.

Step L is conducted in an organic solvent. Suitable solvents include dichloromethane, acetonitrile, THF, DMF or pyridine. A preferred solvent is THF.

The reaction in Step L can suitably be conducted at a temperature in a range of from about 0° C. to about 40° C. and is typically conducted at a temperature in a range of from about 10° C. to about 28° C.

Another embodiment of Process P comprises Steps B to L as described above or Steps A to L as described above, and further comprises:

(M) treating Compound XIII with acid to obtain a compound of Formula XIV:

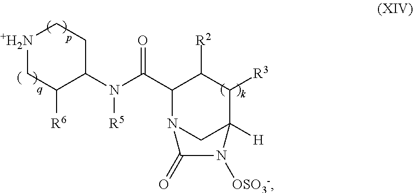

or a salt thereof. Compounds encompassed by Formula XIV can exhibit inhibition of β-lactamase and thus can be used as β-lactamase inhibitors in combination with β-lactam antibiotics (e.g., imipenem, ceftazidime and piperacillin) to treat bacterial infections caused by microorganisms normally resistant to β-lactam antibiotics due to the presence of the β-lactamases. Of particular interest are compounds of Formula XIV in which R² ═R³═H and k=1. The acid treatment removes the Boc protecting group. The acid is suitably a mineral acid, a Lewis acid, or an organic acid. Suitable mineral acids include hydrogen halides (HCl, HBr, and HF, as a gas or in aqueous solution), sulfuric acid, tetrafluoroboric acid and nitric acid. Suitable organic acids include carboxylic acids, alkylsulfonic acids and arylsulfonic acids. Exemplary organic acids include trifluoroacetic acid (TFA), toluenesulfonic acid, benzenesulfonic acid, methanesulfonic acid, and trifluoromethanesulfonic acid. Suitable Lewis acids include BF₃.Et₂O, SnCl₄, ZnBr₂, Me₃SiI, Me₃SiCl, Me₃SiOTf, and AlCl₃. A class of suitable acids consists of Me₃SiOTf, TFA, and tetrafluoroboric acid. A preferred acid is tetrafluoroboric acid. The acid is typically employed in an amount in a range of from about 1.0 to about 2.0 equivalents per equivalent of Compound XI, and is more typically employed in an amount in a range of from about 1.2 to about 1.5 equivalents. The treatment is suitably conducted at a temperature in a range of from about −10° C. to about 25° C. and is typically conducted at a temperature in a range of from about 0° C. to about 10° C.

A sub-embodiment of Process P is a process for preparing Compound 4:

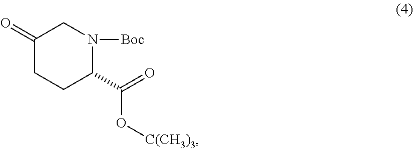

which comprises:

(B) contacting ketosulfoxonium ylide 3:

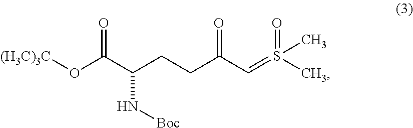

with a catalyst selected from the group consisting of iridium cyclooctadiene chloride dimer ([Ir(COD)Cl]₂), Ir(COD)₂BF₄, and Ir(COD)₂BARF, to obtain Compound 4.

Another sub-embodiment of Process P comprises Step B as just described in the preceding sub-embodiment to obtain Compound 4, and further comprises:

(A) contacting Compound 2:

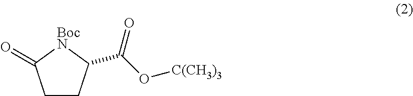

with a trimethylsulfoxonium halide in the presence of a strong base selected from the group consisting of Na C₁₋₄ alkoxides and K C₁₋₄ alkoxides to obtain Compound 3.

Another sub-embodiment of Process P comprises Step B as just described in the above sub-embodiment or Steps A and B as just described in the preceding sub-embodiment, and further comprises:

(C) treating Compound 4 with a reducing agent selected from the group consisting of Li borohydride, Na borohydride and K borohydride, to obtain Compound 5:

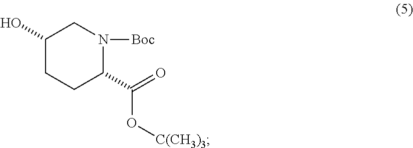

and

(D) contacting Compound 5 with a sulfonyl halide of formula R⁴—SO₂Cl in the presence of a tri-C₁₋₄ alkylamine base to obtain a compound of Formula v:

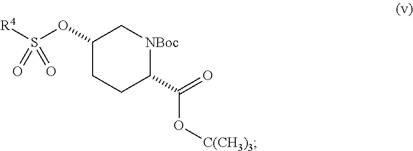

wherein R⁴ is methyl, chloromethyl, phenyl, 4-bromophenyl, 4-trifluoromethylphenyl, 4-methylphenyl, or 2,4-dichlorophenyl. An aspect of this sub-embodiment is the process further comprising Steps C and D as just described, except that R⁴ is methyl, chloromethyl, phenyl, 4-bromophenyl, 4-trifluoromethylphenyl, or 4-methylphenyl.

Another sub-embodiment of Process P comprises Steps B to D or Steps A to D as just described in the preceding sub-embodiment, and further comprises:

(E) treating Compound v with acid selected from the group consisting of hydrochloric acid, sulfuric acid, trifluoroacetic acid, and phosphoric acid to obtain Compound vi:

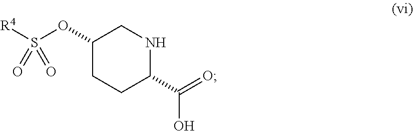

and

(F) treating Compound vi with an Boc-producing agent selected from the group consisting of di-t-butylcarbonate and Boc-ON to obtain a Compound vii:

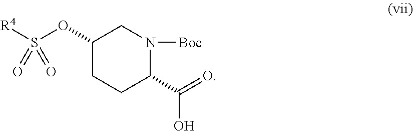

Another sub-embodiment of Process P comprises Steps A to F or Steps B to F as just described in the preceding sub-embodiment, and further comprises:

(G) contacting Compound vii with an amine selected from the group consisting of:

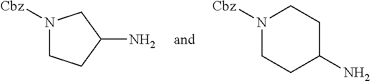

in the presence of a coupling agent to obtain an amide of Formula viii:

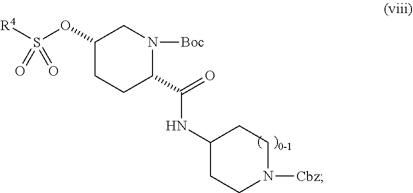

wherein the coupling agent is selected from the group consisting of DCC or EDC.

Another sub-embodiment of Process P comprises Steps A to G or Steps B to G as just described in the preceding sub-embodiment, and further comprises:

(H) contacting Compound viii with N-Boc-O-benzylhydroxylamine in the presence of a base selected from the group consisting of Li t-butoxide, Na t-butoxide, K t-butoxide, K amyloxide and cesium carbonate (and preferably selected from K t-butoxide and cesium carbonate) to obtain Compound ix:

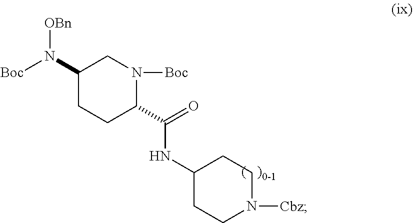

and

(I) treating Compound ix with an acid selected from the group consisting of methanesulfonic acid, chloromethanesulfonic acid, p-toluenesulfonic acid and benzenesulfonic acid to obtain Compound x:

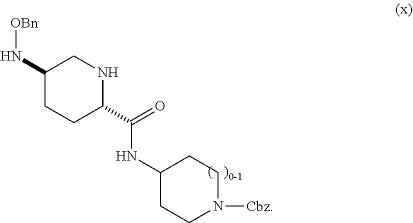

Another sub-embodiment of Process P comprises Steps A to I or Steps B to I as just described in the preceding sub-embodiment, and further comprises:

(J) contacting Compound x with triphosgene in the presence of a tri-C₁₋₄ alkylamine base, and then adding an aqueous solution of phosphoric acid to obtain Compound xi:

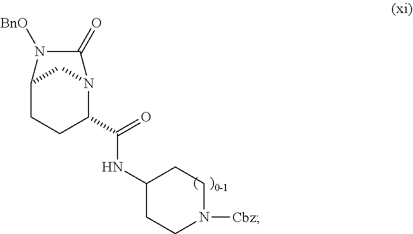

and

(K) contacting Compound xi with hydrogen in the presence of a Pd catalyst and a Boc-producing agent selected from the group consisting of di-t-butylcarbonate and Boc-ON to obtain Compound xii:

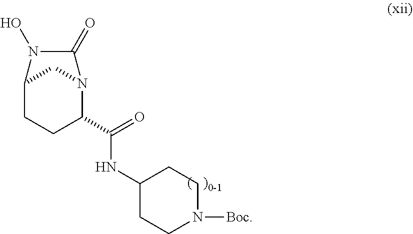

Another sub-embodiment of Process P comprises Steps A to K or Steps B to K as just described in the preceding sub-embodiment, and further comprises:

(L) contacting Compound xii with a sulfating agent selected from the group consisting of pyridine-SO₃ complex, chlorosulfonic acid and DMF-SO₃ complex in the presence of 2-picoline to obtain Compound xiii:

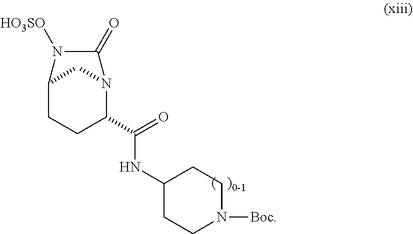

Another sub-embodiment of Process P comprises Steps A to L or Steps B to L as just described in the preceding sub-embodiment, and further comprises:

(M) treating Compound xiii with acid to obtain Compound xiv:

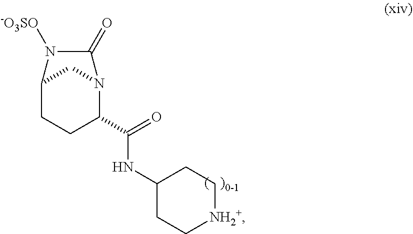

or a salt thereof.

The solvents, agents, catalysts, reaction amounts, reaction temperatures, etc. described above for Steps A to M in Process P leading to Compound XIV are applicable to Steps A to M set forth in the preceding sub-embodiments leading to Compound xiv, except where express limitations are placed upon one or more of these variables in the sub-embodiments. For example, the sub-embodiment of Process P describing the preparation of Compound 4 from Compound 3 restricts the catalyst employed in Step B to a specific group of Ir catalysts. Accordingly, the broader disclosure of suitable catalysts provided for in Process P as originally set forth above does not apply to this sub-embodiment.

It is to be understood that the solvents, agents, catalysts, reaction amounts, reaction temperatures, etc. described above with respect to Process P and its embodiments and sub-embodiments are intended only to illustrate, not limit, the scope of the process. For example, the solvent employed in any of Steps A to M can be any organic substance which under the reaction conditions employed in the step of interest is in the liquid phase, is chemically inert, and will dissolve, suspend, and/or disperse the reactants and any reagents so as to bring the reactants and reagents into contact and to permit the reaction to proceed. Similar considerations apply to the choice of bases, catalysts, and other reagents employed in the process steps. Furthermore, each of the steps can be conducted at any temperature at which the reaction forming the desired product can detectably proceed. The reactants, catalysts and reagents in a given step can be employed in any amounts which result in the formation of at least some of the desired product. Of course, a high conversion (e.g., at least about 60% and preferably higher) of starting materials in combination with a high yield (e.g., at least about 50% and preferably higher) of desired products is typically the objective in each step, and the choice of solvents, agents, catalysts, reaction amounts, temperatures, etc. that can provide relatively good conversions and yields of product are preferred, and the choices that can provide optimal conversions and yields are more preferred. The particular solvents, agents, catalysts, reaction amounts, reaction temperatures, etc. described above with respect to Process P and its embodiments and sub-embodiments can provide good to optimum conversions and yields.

The reaction times for the process steps described above depend upon such factors as (i) the choice and relative proportions of the starting substrate and other reagents, (ii) the choice of solvent, (iii) the choice of reaction temperature, and (iv) the level of conversion desired. The reactions are typically conducted for a time sufficient to achieve 100% conversion.

The progress of any reaction step set forth herein can be followed by monitoring the disappearance of a reactant (e.g., Compound II in Step B) and/or the appearance of the desired product (e.g., Compound III in Step B) using such analytical techniques as TLC, HPLC, IR, NMR or GC.

The present invention also includes a method for purifying compound 11:

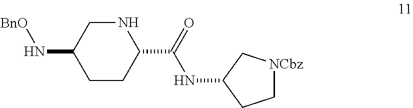

which comprises adding an antisolvent to a solution of compound 11 and di-p-toluoyl-L-tartaric acid in an organic solvent to form a suspension of crystals of the di-p-toluoyl-L-tartaric acid salt of 11, and then recovering the crystals. In a preferred embodiment, the organic solvent is acetonitrile, the antisolvent is IPAc, and the crystals are recovered by separating the crystals from the supernatant (e.g., by filtration) and then drying the separated crystals (e.g., in a vacuum oven with nitrogen sweep). Seed crystals can be added during or after the addition of the antisolvent to reduce crystallization time and/or to improve the consistency and yield of the crystals, but seed is not required. Additional amounts of solvent and antisolvent can be added to the suspension to reduce the thickness of the suspension to permit more efficient stirring and easier handling. Purified 11 can be obtained by treating the crystalline salt with base (e.g., NaHCO₃) and recovering 11.

The present invention also includes another method for purifying compound

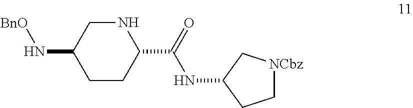

which comprises adding aqueous hydrochloric acid to a solution of compound 11 in an organic solvent to form a suspension of crystals of the HCl salt of 11, and then recovering the crystals. In a preferred embodiment, the organic solvent is 2-propanol and the crystals are recovered by separating the crystals from the supernatant (e.g., by filtration) and then drying the separated crystals (e.g., in a vacuum oven with nitrogen sweep). Seed crystals can be added during or after the addition of the hydrochloric acid to reduce crystallization time and/or to improve the consistency and yield of the crystals, but seed is not required. Additional amounts of solvent can be added to the suspension to reduce the thickness of the suspension to permit more efficient stirring and easier handling. Solvent may be distilled from the slurry to azeotropically remove water to improve recovery of the crystals. Purified 11 can be obtained by treating the crystalline salt with base (e.g., NaHCO₃) and recovering 11.

The present invention also includes a process (alternatively referred to as Process Q) for preparing a compound of Formula XIII-Es:

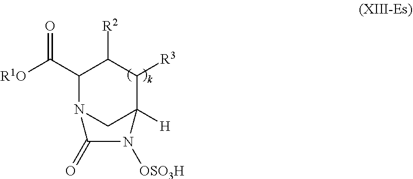

wherein the process comprises the synthetic steps set forth above in Process P, except that the steps involving the formation and/or protection of the amide side chain are excluded. Thus, the process for preparing Compound XIII-Es comprises Steps A, B, C, D, H, I, J, K′ (identical to K except the Boc-producing agent is absent), and L:

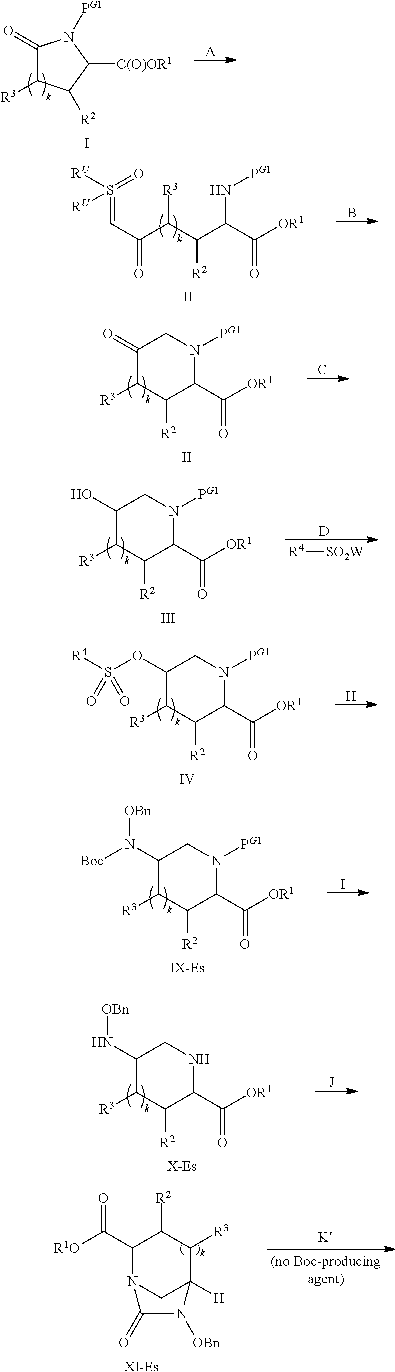

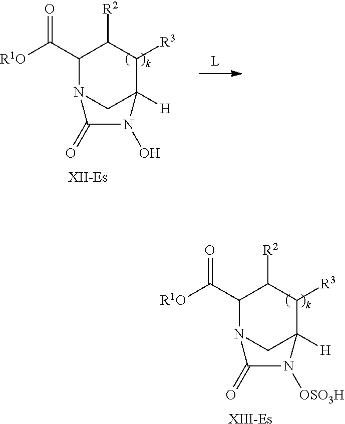

The descriptions of these steps as set forth above in the discussion of Process P also apply to Process Q.

Unless expressly stated to the contrary, all ranges cited herein are inclusive; i.e., the range includes the values for the upper and lower limits of the range as well as all values in between. For example, a phenyl ring described as optionally substituted with “1 to 3 substituents” is intended to include as aspects thereof; a ring substituted with 1 to 3 substituents, 2 to 3 substituents, 3 substituents, 1 to 2 substituents, 2 substituents, and 1 substituent. As another example, temperature ranges, ranges of equivalents, and the like described herein include the upper and lower limits of the range and any value in the continuum therebetween.

The term “alkyl” refers to a monovalent straight or branched chain, saturated aliphatic hydrocarbon radical having a number of carbon atoms in the specified range. Thus, for example, “C₁₋₆ alkyl” (or “C₁-C₆ alkyl”) refers to any of the hexyl and pentyl alkyl isomers as well as n-, iso-, sec- and t-butyl, n- and iso-propyl, ethyl and methyl. As another example, “C₁₋₄ alkyl” refers to n-, iso-, sec- and t-butyl, n- and isopropyl, ethyl and methyl. As another example, “C₁₋₃ alkyl” refers to n-propyl, isopropyl, ethyl and methyl.

The term “branched alkyl” refers to an alkyl group as defined above except that straight chain alkyl groups in the specified range are excluded. As defined herein, branched alkyl includes alkyl groups in which the alkyl is attached to the rest of the compound via a secondary or tertiary carbon; e.g., isopropyl is a branched alkyl group.

The term “halogen” (or “halo”) refers to fluorine, chlorine, bromine and iodine (alternatively referred to as fluoro, chloro, bromo, and iodo).

The term “haloalkyl” refers to an alkyl group as defined above in which one or more of the hydrogen atoms have been replaced with a halogen (i.e., F, Cl, Br and/or I). Thus, for example, “C₁₋₄ haloalkyl” (or “C₁-C₄ haloalkyl”) refers to a C₁ to C₄ linear or branched alkyl group as defined above with one or more halogen substituents. The term “fluoroalkyl” has an analogous meaning except that the halogen substituents are restricted to fluoro. Suitable fluoroalkyls include the series (CH₂)₀₋₄CF₃ (i.e., trifluoromethyl, 2,2,2-trifluoroethyl, 3,3,3-trifluoro-n-propyl, etc.)

The present invention also includes a compound selected from the group consisting of:

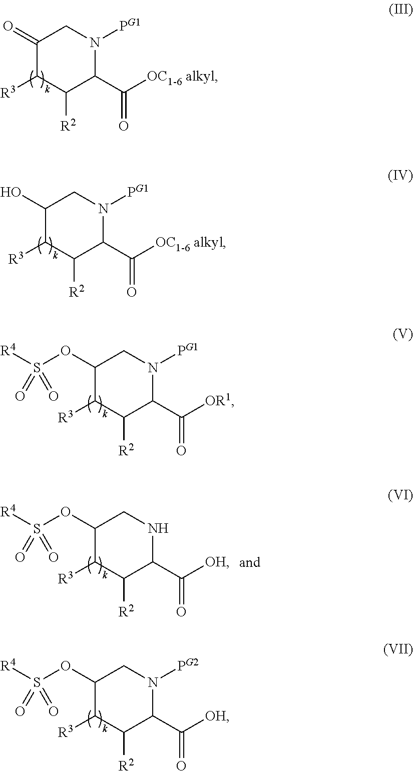

wherein:
P^{G1} is an amine protective group which forms with the amino nitrogen to which it is attached a carbamate or a benzylamine;
P^{G2} is an amine protective group which forms with the amino nitrogen to which it is attached an alkyl carbamate;
R¹ is C₁₋₆ alkyl or C₁₋₆ alkyl mono- or di-substituted with AryA, wherein each AryA is independently phenyl or napthyl and is optionally substituted with from 1 to 3 substituents each of which is independently halogen, C₁₋₆ alkyl, or O—C₁₋₆ alkyl;
k is an integer equal to 0, 1 or 2;
R² and R³ are defined as follows:
(a) R² is H, C₁₋₆ alkyl, O—C₁₋₆ alkyl, or Si(—C₁₋₆ alkyl)₃; and each R³ is H or C₁₋₆ alkyl; or
(b) alternatively and with the proviso that k is 1 or 2, R² and the R³ adjacent to R² together with the carbon atoms to which each is attached form C₅₋₇ cycloalkyl which is optionally substituted with from 1 to 3 substituents each of which is independently C₁₋₆ alkyl, O—C₁₋₆ alkyl, O—Si(—C₁₋₆ alkyl)₃, or O—Si(—C₁₋₆ alkyl)(-phenyl)₂; and any other R³ is H or C₁₋₆ alkyl.

### R⁴ is:

(1) phenyl optionally substituted with from 1 to 3 substituents each of which is independently C₁₋₄ alkyl, C₁₋₄ haloalkyl, O—C₁₋₄ alkyl, O—C₁₋₄ haloalkyl, Cl, Br, F, or NO₂;
(2) C₁₋₄ alkyl; or
(3) C₁₋₄ haloalkyl.

The present invention also includes a compound selected from the group consisting of:

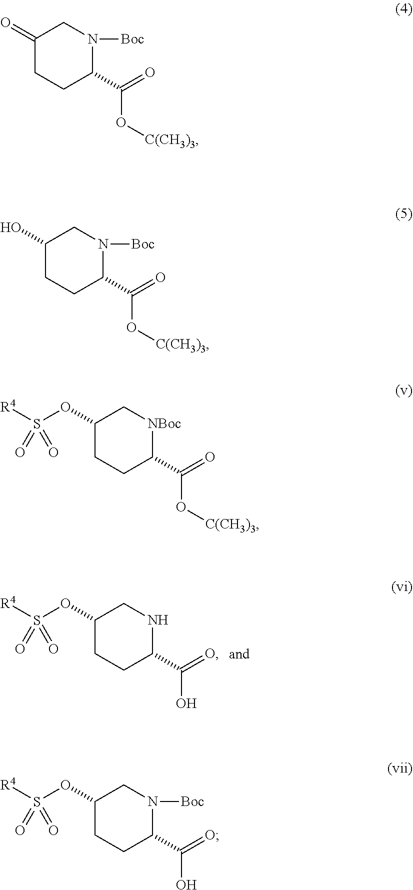

wherein R⁴ is methyl, chloromethyl, phenyl, 4-bromophenyl, 4-trifluoromethylphenyl, 4-methylphenyl, or 2,4-dichlorophenyl. A sub-class of interest includes the compounds of formula 4, 5, v, vi, and vii in which R⁴ is methyl, chloromethyl, phenyl, 4-bromophenyl, 4-trifluoromethylphenyl, or 4-methylphenyl.

Abbreviations employed herein include the following:

acac=acetylacetonate;
BARF=the tetra-aryl borate non-coordinating anion of formula [B[3,5-(CF₃)₂C₆H₃]₄]⁻;
BLI=beta-lactamase inhibitor;
Bn=benzyl;
Boc=t-butyloxycarbonyl;
Boc-ON=2-(tert-butoxycarbonyloxyamino)-2-phenyl acetonitrile;

### Boc-OSN=N-tert-butoxycarbonyloxy)succinimide;

Boc₂O=di-t-butyl carbonate;
BOP=benzotriazol-1-yloxytris-(dimethylamino)phosphonium;
Cbz=carbobenzoxy (alternatively, benzyloxycarbonyl);
COD=cyclooctadienyl;
DCC=dicyclohexyl carbodiimide;
DCE=1,2-dichloroethane;
DCM=dichloromethane;
DIPEA=diisopropylethylamine (or Hunig's base);

### DMAC=N,N-dimethylacetamide;

DMAP=4-dimethylaminopyridine N,N-dimethylaminopyridine;
DME=1,2-dimethoxyethane;

### DMF=N,N-dimethylformamide;

DMSO=dimethyl sulfoxide;
DPPA=diphenylphosphoryl azide
EDC=1-ethyl-3-(3-dimethylaminopropyl) carbodiimide;
Et=ethyl;
EtOAc=ethyl acetate;
GC=gas chromatography;
HATU=O-(7-Azabenzotriazol-1-yl)N,N,N′,N′-tetramethyluronium hexafluorophosphate;
HMDS=hexamethyldisilazide;
HOAt=1-hydroxy-7-azabenzotriazole;
HOBt=1-hydroxy benzotriazole;
HOPO=2-hydroxypyridine-N-oxide;
HPLC=high-performance liquid chromatography;
i-Pr=isopropyl;
IPA=isopropyl alcohol;
IPAc=isopropyl acetate;
IR=infrared
MeOH=methanol;

### NMP=N-methylpyrrolidinone;

NMR=nuclear magnetic resonance
P^{G}=protective group;
PyBOP=benzotriazol-1-yl-oxytripyrrolidinophosphonium hexafluorophosphate;
TBTU=2-(1H-benzotriazole-1-yl)-1,1,3,3-tetramethyluronium tetrafluoroborate;
t-Bu=tert-butyl;
TEA=triethylamine;
TFA=trifluoroacetic acid;
TFE=2,2,2-trifluoroethanol;
THF=tetrahydrofuran;
TLC=thin layer chromatography.

The following examples serve only to illustrate the invention and its practice. The examples are not to be construed as limitations on the scope or spirit of the invention.

### Example 1

### Sulfuric acid mono-[(2S,5R)-7-oxo-2-((S)-pyrrolidin-3-ylcarbamoyl)-1,6-diaza-bicyclo[3.2.1]oct-6-yl]ester

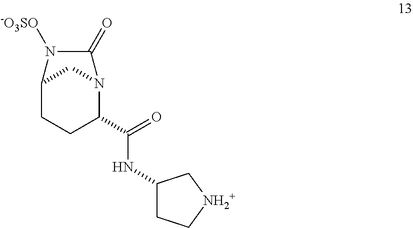

### Step 1: (S)-5-Oxo-pyrrolidine-2-carboxylic acid tert-butyl ester (1)

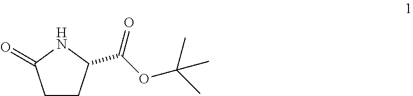

To a 2 L 3-neck round bottom flask equipped with overhead stirring, nitrogen inlet, and thermocouple was charged L-pyroglutamic acid (40 g., 310 mmol), DCM (400 mL), and H₂SO₄ (16.51 mL, 310 mmol) the resulting slurry was cooled to 0° C. Meanwhile 145 mL (1549 mmol) of isobutylene was condensed and added to the DCM slurry over 3 minutes; a slight exotherm was observed. The slurry became thicker after addition of isobutylene. The reaction was allowed to warm to room temperature over 1 hour. A cold finger with dry-ice/acetone was put in place to re-condense any gaseous isobutylene. The reaction was left at room temperature overnight. After the overnight age the reaction became homogenous and colorless. The reaction was poured into 350 mL of 0.5N NaOH and 400 mL IPAc. Once the reaction was quenched the aqueous layer was checked to make sure the pH was at least 10. The aqueous layer was removed and the organics were dried over MgSO₄ then filtered and concentrated to give (S)-5-Oxo-pyrrolidine-2-carboxylic acid tert-butyl ester as an off-white solid (44 g., 241 mmol, 78%). ¹H NMR (500 MHz, CDCl₃): δ 6.05 (br s, 1H), 4.15 (m, 1H), 2.3-2.5 (m, 3H), 2.2 (m, 1H) 1.5 (S, 9H).

### Step 2: (S)-5-Oxo-pyrrolidine-1,2-dicarboxylic acid di-tert-butyl ester (2)

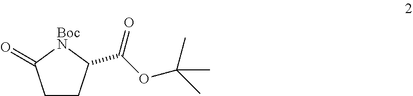

To a 1-neck 1 L round bottom flask was charged 40 g (216 mmol) (S)-5-oxo-pyrrolidine-2-carboxylic acid tert-butyl ester followed by 350 mL MeCN. The reaction was cooled to 5° C. followed by addition of DMAP (0.5 g., 4.32 mmol) and Boc₂O (47.1 g., 216 mmol). The reaction was allowed to warm to room temperature over 30 minutes. After 1.5 hours TLC indicated the reaction was complete. Water (300 mL) and IPAc (400 mL) were added and the solution was transferred to a 2 L separatory funnel. The aqueous layer was cut and the organics dried over MgSO₄ then filtered and concentrated to an oil. The oil was taken up in minimal amount of EtOAc and chromatographed using 600 g of silica on a linear gradient from 100% hexanes to 1:1 hexanes:EtOAc. The collected fractions were concentrated to an oil to give 60 g (210 mmol, 97% yield) of (S)-5-oxo-pyrrolidine-1,2-dicarboxylic acid di-tert-butyl ester. ¹H NMR (500 MHz, CDCl₃): δ 4.5 (dd, J=2.5, 6.85 Hz, 1H), 2.4-2.7 (m, 2H), 2.3 (m, 1H), 2.0 (m, 1H), 1.57 (5, 9H), 1.50 (S, 9H).

### Step 3: (S)-3-((S)-2-tert-Butoxycarbonylamino-6-dimethylsulfoxonium-5-oxo-hexanoylamino)-pyrrolidine-1-carboxylic acid tert-butyl ester (3)

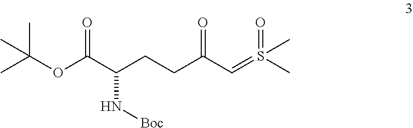

To a 1 L 3-neck round bottom flask equipped with overhead stirring and nitrogen inlet was charged trimethylsulfoxonium iodide (56 g., 249 mmol) and dry DMSO (250 mL). To the resultant slurry was added KOtBu (17.78 g., 240 mmol) in three portions over 15 minutes. Over the next hour the orange slurry turned to a colorless homogeneous solution. The (S)-5-oxo-pyrrolidine-1,2-dicarboxylic acid di-tert-butyl ester (50.7 g., 178 mmol) was added slowly over 10 minutes using a DMSO rinse at room temperature. After one hour the reaction was complete, as shown by TLC. 1 L water was added (exotherm observed) then 500 mL EtOAc and the resulting solution aged for 10 minutes. The biphasic solution was transferred to a separatory funnel and the aqueous layer was removed. The organics were washed with 500 mL water and the layers were cut. The organic layer was dried over MgSO₄ then filtered and concentrated to provide (S)-3-(S)-2-tert-Butoxycarbonylamino-6-dimethylsulfoxonium-5-oxo-hexanoylamino)-pyrrolidine-1-carboxylic acid tert-butyl ester as a light yellow solid (67 g., 177 mmol, 100% yield). ¹H NMR (500 MHz, DMSO-d₆): δ 7.15 (d, J=7.4 Hz, 1H), 4.7 (S, 1H), 3.6 (m, 1H), 3.4, (S, 1H), 2.0-2.15, (m, 2H), 1.5, (m, 1H), 1.7, (m, 1H), 1.4 (5, 18H).

### Step 4: (S)-5-Oxo-piperidine-1,2-dicarboxylic acid di-text-butyl ester (4)

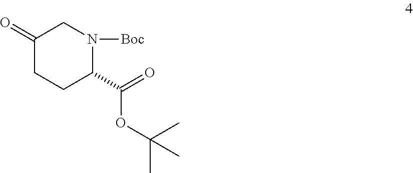

To a 3 L 3-neck round bottom flask equipped with overhead stirring, thermocouple, and nitrogen inlet was charged 1200 mL of thoroughly degassed DCE and Ir(COD)₂Cl₂ (2.2 g., 7.10 mmol) and heated to 80° C. Meanwhile the (S)-3-(S)-2-tert-butoxycarbonylamino-6-dimethylsulfoxonium-5-oxo-hexanoylamino)-pyrrolidine-1-carboxylic acid tert-butyl ester (67 g., 177 mmol) was taken up in 500 mL of degassed DCE and transferred to an addition funnel. The ylide was added via addition funnel over 3 hours. The reaction was aged at 80° C. overnight. After overnight age TLC showed reaction was complete. The orange homogeneous solution was concentrated to an oil and taken on to the next step without further purification or quantification.

### Step 5: (2S,5S)-5-Hydroxy-piperidine-1,2-dicarboxylic acid di-tert-butyl ester (5)

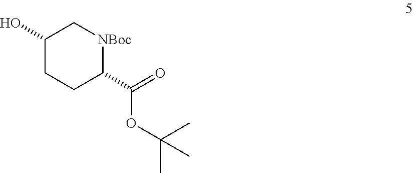

To a 1 L 3-neck round bottom flask equipped with overhead stirring, nitrogen inlet, and thermocouple was charged 264 mL THF, 3 mL MeOH, and 73.5 mL of 2M LiBH₄ in THF (147 mmol) at room temperature and aged for 30 minutes. The solution was then cooled to −10° C. and (S)-5-oxo-piperidine-1,2-dicarboxylic acid di-tert-butyl ester was added as a 4 mL/g solution in THF (44 g., 147 mmol, 176 mL THF) keeping the internal temperature below −5° C. The addition took 40 minutes. After one hour at 0° C. TLC showed the reaction to be complete. Meanwhile a 20% acetic acid in MeOH solution was prepared by adding 40 mL of acetic acid to 160 mL MeOH. This solution was transferred to an addition funnel. While keeping the internal temperature below 0° C. 20 mL of the acetic acid/MeOH solution was added watching for excess gas evolution. The solution was aged for 30 minutes, and then warmed to room temperature at which point the rest of the acetic acid/MeOH solution was added keeping the internal temperature below 25° C. The mixture was then aged for one hour. Water (500 mL) and IPAc (500 mL) was added and transferred to a 2 L separatory funnel. The aqueous layer was cut and the organics was washed twice with 500 mL water and once with 500 mL saturated sodium bicarbonate. The organics were dried with MgSO₄ and concentrated to a dark tan oil and taken on to the next step without further purification. NMR with internal standard showed 35 g. (116 mmol, 79% yield over two steps) of alcohol. ¹H NMR (500 MHz, CDCl₃): δ 4.75 (bs, 0.5H), 4.55 (bs, 0.5H), 4.05-4.3 (m, 1H), 3.65 (bs, 1H), 2.75 (m, 1H), 2.3 (bs, 1H), 2.0 (m, 1H), 1.7 (bt, 1H), 1.5 (s, 19H).

### Step 6: (2S,5S)-5-(4-Trifluoromethyl-benzenesulfonyloxy)-piperidine-1,2-dicarboxylic acid di-tert-butyl ester (6)

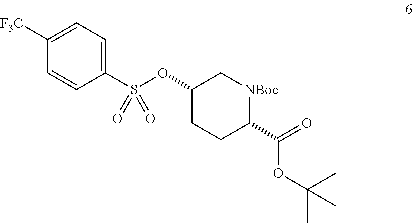

To a 1 L 3-neck round bottom flask equipped with overhead stirring, nitrogen inlet, and thermocouple was charged (2S,5S)-5-hydroxy-piperidine-1,2-dicarboxylic acid di-tert-butyl ester (36 g., 119 mmol) in 350 mL of DCM, TEA (50 mL, 358 mmol), and DMAP (0.146 g., 1.2 mmol). 4-(trifluoromethyl)-benzenesulfonyl chloride (38 g., 155 mmol) was taken up in 50 mL DCM and slowly added to the reaction mixture keeping the internal temperature below 25° C. The reaction was allowed to age overnight at 25° C. The reaction was complete by TLC after overnight age. Water (400 mL) was added to the reaction mixture and the biphasic mixture transferred to a separatory funnel. The aqueous layer was cut and the organic layer washed with water (400 mL) 2× and 1N HCl 300 mL 1×. The organics were then dried over MgSO₄, then filtered and concentrated to a dark tan oil. This oil was run through a silica plug (300 g) with 3:1 EtOAc:hexanes as the eluant to remove much of the color. The organics were then concentrated to provide (2S,5S)-5-(4-trifluoromethyl-benzenesulfonyloxy)-piperidine-1,2-dicarboxylic acid di-tert-butyl ester as a light yellow oil (assay yield: 55 g., 90%). This oil was taken on to the next step without further purification. ¹H NMR (500 MHz, CDCl₃): δ 8.1 (m, 2H), 7.85 (m, 2H), 4.6-4.8 (m, 0.5H), 4.4-4.6 (m 1.5H), 4.2 (m, 0.5H), 4.0 (m, 0.5H), 2.9 (m, 1H), 2.3 (m, 1H), 2.0-2.2 (m, 2H), 1.7 (m, 1H), 1.4 (s, 18H).

### Step 7: (2S,5S)-5-(4-Trifluoromethyl-benzenesulfonyloxy)-piperidine-2-carboxylic acid (7)

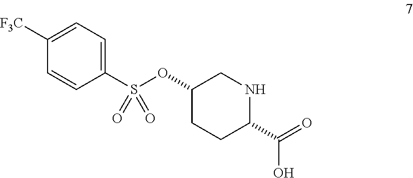

In a 1 L 3 neck round bottom flask equipped with overhead stirring, nitrogen inlet, and thermocouple was charged (2S,5S)-5-(4-trifluoromethyl-benzenesulfonyloxy)-piperidine-1,2-dicarboxylic acid di-tert-butyl ester (55 g., 108 mmol) in DCM (200 mL). Trifluororacetic acid (125 mL, 1619 mmol) was added over 5 minutes keeping the temperature below 25° C. The reaction was aged overnight to achieve full conversion. The reaction was complete after overnight age as determined by HPLC. The TFA was then removed under reduced pressure with 5× 600 mL DCE additions to help azeotrope the TFA. The reaction was then taken up in DCM (500 mL) and moved to the next step without further purification or quantification.

### Step 8: (2S,5S)-5-(4-Trifluoromethyl-benzenesulfonyloxy)-piperidine-1,2-dicarboxylic acid 1-tert-butyl ester (8)

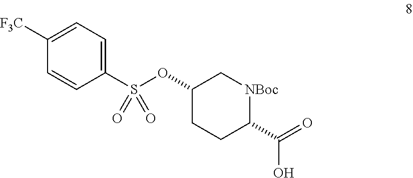

In a 1 L 3-neck round bottom flask equipped with overhead stirring, nitrogen inlet and thermocouple was charged the DCM/(2S,5S)-5-(4-Trifluoromethyl-benzenesulfonyloxy)-piperidine-2-carboxylic acid solution from intermediate 7. The solution was cooled with a ice/acetone bath to −10° C. Triethylamine (60 mL, 432 mmol) was added very slowly keeping the internal temperature below −5° C. Once all the triethylamine was added, Boc₂O (23 g., 108 mmol) was charged in three portions keeping the internal temperature below 0° C. DMAP (0.132 g., 1.08 mmol) was then added in one portion and the reaction was allowed to warm to room temperature. The reaction was judged complete by HPLC after 30 min and quenched with the addition of 500 mL water and 200 mL DCM. The pH of the aqueous layer should be close to 10 and is necessary to remove excess TFA from the previous reaction. The aqueous layer is removed and the organics are dried with MgSO₄ then filtered and concentrated to a volume of 100 mL.

### Step 9: (2S,5S)-2-((S)-1-Benzyloxycarbonyl-pyrrolidin-3-ylcarbamoyl)-5-(4-trifluoromethyl-benzenesulfonyloxy)-piperidine-1-carboxylic acid tert-butyl ester (9)

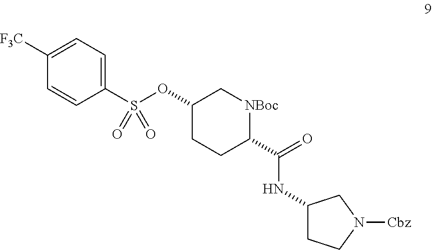

In a 1 L 3-neck round bottom flask equipped with overhead stirring, nitrogen inlet, and thermocouple was charged 400 mL DCM followed by (S)-(+)-1-Cbz-3-aminopyrrolidine HCl salt (28 g., 106 mmol) and TEA (14.8 mL, 106 mmol) the resultant slurry was aged for 30 minutes. After the 30 minute age, the (2S,5S)-5-(4-trifluoromethyl-benzenesulfonyloxy)-piperidine-1,2-dicarboxylic acid 1-tert-butyl ester in DCM (100 mL) was added in one portion followed by HOPO (1.2 g., 11 mmol) and EDC in three portions (19.72 g., 127 mmol). The reaction was aged at room temperature for 3 hours. After 3 hours the reaction was complete by HPLC and quenched by the addition of 400 mL of 1N HCl and aged for 10 minutes. The contents were transferred to a 2 L separatory funnel where the aqueous layer was removed. The organics were dried with MgSO₄ and filtered. The solvent was then switched from DCM to MTBE. Once all the DCM was removed the volume of MTBE was adjusted to 500 mL and the solution was transferred to a 1 L 3-neck flask with overhead stirring and thermocouple. At room temperature heptanes was added until a seed bed started to form. Once the seed bed began to form, the suspension was aged for 20 minutes, and then 250 mL heptanes was added over 20-30 minutes. The resultant slurry was then aged for 2 hours. The slurry was then filtered and washed with 100 mL 3:1 heptanes:MTBE and dried under vacuum with nitrogen sweep overnight to give 33 g (47% yield over three steps) of (2S,5S)-2-((S)-1-benzyloxycarbonyl-pyrrolidin-3-ylcarbamoyl)-5-(4-trifluoromethyl-benzenesulfonyloxy)-piperidine-1-carboxylic acid tert-butyl ester as an off white solid. ¹H NMR (500 MHz, CDCl₃): δ 8.1 (d, J=8.3 Hz, 2H), 7.88 (d, J=8.3 Hz, 2H), 7.35 (s, 5H), 5.15 (s, 2H), 4.6 (bs, 1H), 4.5 (m, 2FI), 4.2 (m, 1H), 3.75 (m, 1H), 3.5 (m, 2H), 3.3 (m, 1H), 2.3 (t, J=12.7 Hz, 1H), 2.35 (m, 1H), 2.2 (bs, 1H), 1.9 (bs, 1H), 1.8 (bs, 1H), 1.65 (m, 2H), 1.45 (s, 9H).

The following compound was also obtained using the procedure set forth in the preceding paragraph by replacing (S)-(+)-1-Cbz-3-aminopyrrolidine HCl salt with 1-Cbz-4-aminopiperidine HCl salt:

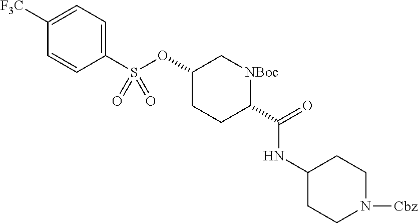

### Step 10: (2S,5R)-5-Benzyloxyamino(carboxylic acid tert-butyl ester)-2-((S)-1-benzyloxycarbonyl-pyrrolidin-3-ylcarbamoyl)-piperidine-1-carboxylic acid tert-butyl ester (10)

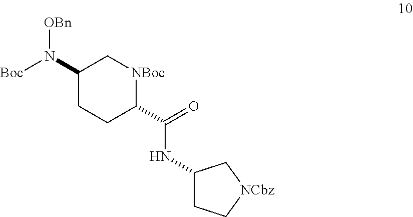

In a 5 L 3-neck round bottom flask equipped with overhead stirring, nitrogen inlet, and thermocouple was charged 1.8 L acetonitrile followed by (2S,5S)-2-((S)-1-Benzyloxycarbonyl-pyrrolidin-3-ylcarbamoyl)-5-(4-trifluoromethyl-benzenesulfonyloxy)-piperidine-1-carboxylic acid tert-butyl ester (236.6 g., 361 mmol) and N-Boc-β-benzylhydroxylamine (101 g., 451 mmol). To this mixture was added cesium carbonate (147 g., 451 mmol), and the resultant slurry was warmed to 65° C. The reaction was aged at this temperature for 6.5 hours. At this time the reaction was complete by HPLC, and was diluted with 2.5 L EtOAc. The contents were transferred to a 6 L separatory funnel where the organic layer was washed three times with 800 mL 5% NaHCO₃, then washed with 600 mL water. The organics were dried with MgSO₄ and filtered. The resulting solution was concentrated to a viscous oil and taken forward to the next step.

### Step 11: (S)-3-[((2S,5R)-5-Benzyloxyamino-piperidine-2-carbonyl)-amino]-pyrrolidine-1-carboxylic acid benzyl ester (11), di-p-toluoyl-L-tartaric acid salt

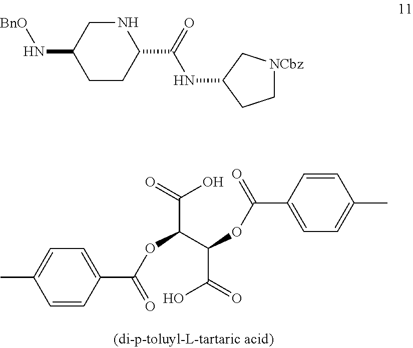

In a 5 L 3-neck round bottom flask equipped with overhead stirring, nitrogen inlet, and a thermocouple was charged 2.0 L DCM followed by (2S,5R)-5-benzyloxyamino(carboxylic acid tert-butyl ester)-2-((S)-1-benzyloxycarbonyl-pyrrolidin-3-ylcarbamoyl)-piperidine-1-carboxylic acid tert-butyl ester (212 g., 325 mmol) followed by slow addition of methanesulfonic acid (156 g., 1625 mmol), keeping internal temperature below 35° C., and the resultant solution was warmed to 40° C. The reaction was aged at this temperature for 1 hour. At this time the reaction was complete by HPLC and was cooled to 10° C. The solution was slowly transferred to a 6 L separatory funnel containing 1 L 5N NaOH. After mixing the aqueous and organic layers for 2 minutes, the organic layer was removed, and the aqueous layer was washed with 1 L DCM. The combined organics were concentrated to an oil, and this oil was purified via forced flow column chromatography on silica gel using a linear gradient of eluant starting from 100% DCM and progressing to 10% MeOH/1% NH₄OH/89% DCM. The resulting (S)-3-[((2S,5R)-5-benzyloxyamino-piperidine-2-carbonyl)-amino]-pyrrolidine-1-carboxylic acid benzyl ester still had some residual impurity and was further purified by crystallization as its di-p-toluoyl-L-tartaric acid salt as follows: The (S)-3-[((2S,5R)-5-benzyloxyamino-piperidine-2-carbonyl)-amino]-pyrrolidine-1-carboxylic acid benzyl ester (37.1 g, 82 mmol) was dissolved in acetonitrile (111 ml, 2125 mmol) in a 2-L RBF. Di-p-toluoyl-L-tartaric acid (32.3 g, 84 mmol) was added, leading to formation of a thick oil. IPAc (222 mL) was added, and the suspension was heated to turn over the oil to crystals. Addition of seed crystals and vigorous stirring afforded this turnover, yielding a very thick crystalline suspension. Additional IPAc (240 mL) and acetonitrile (37 mL) were added to help stirring (12.5 vol IPAc:4 vol ACN). Supernatant assay at this point showed 11.3 g (30.4%) of diamine in the supernatant, and a 1.5:1 ratio of tartaric acid:diamine. IPAc (280 mL, 7.5 vol) was added (20:4). Supernatant assay showed 8.45 g (22.8%) of diamine in the supernatant, and a 1.7:1 ratio of tartaric acid:diamine. IPAc (300 mL, 8 vol) was added (28:4). Supernatant assay showed 7.55 g (20.4%) of diamine in the supernatant. The slurry was filtered and washed with 9:1 isopropyl acetate:acetonitrile. The resulting crystals were dried at 40° C. in a vacuum oven wth nitrogen sweep to provide (S)-3-[((2S,5R)-5-benzyloxyamino-piperidine-2-carbonyl)-amino]-pyrrolidine-1-carboxylic acid benzyl ester di-p-toluoyl-L-tartaric acid salt (54.5 g., 65 mmol). ¹H NMR (400 MHz, DMSO-d₆): δ 8.42 (s, 1H), 7.79 (d, 4H, J=8.2 Hz), 7.30-7.39 (m, 10H), 7.27 (d, 4H, J=8.1 Hz), 5.60 (s, 2H), 5.08 (d, 2H, J=3.6 Hz), 4.59 (s, 2H), 4.25 (s, 1H), 2.99-3.58 (m, 11H), 2.35 (s, 6H), 1.72-2.12 (m, 4H), 1.37-1.55 (m, 1H), 1.17-1.31 (m, 1H) ppm

### Step 11A (S)-3-[((2S,5R)-5-Benzyloxyamino-piperidine-2-carbonyl)-amino]-pyrrolidine-1-carboxylic acid benzyl ester (11), HCl salt

The (S)-3-[((2S,5R)-5-benzyloxyamino-piperidine-2-carbonyl)-amino]-pyrrolidine-1-carboxylic acid benzyl ester (1.384 g, 3.06 mol) obtained as above was dissolved in 2-propanol (10 L) and the solution was heated to 40° C. and a freshly titrated solution of 5-6 N HCl in 2-propanol (2.1 eq) was added. The resulting exothermic reaction caused a rise in temperature to 50° C. The solution was then allowed cool to 20° C. giving a thin slurry. Solvent was distilled under vacuum to reduce the water content to <1 g/L, while adding dry solvent such as to maintain constant volume (˜13 L). The supernatant assay showed 69 g of product (5%). The slurry was then filtered and the cake was washed with 2-propanol and dried under N2 giving 1310 g HCl salt (82% yield). The crystalline HCl salt can be employed in the next step in place of di-p-toluoyl-L-tartaric acid salt.

### Step 12: (S)-3-[((2S,5R)-6-Benzyloxy-7-oxo-1,6-diaza-bicyclo[3.2.1]octane-2-carbonyl)-amino]-pyrrolidine-1-carboxylic acid benzyl ester (12)

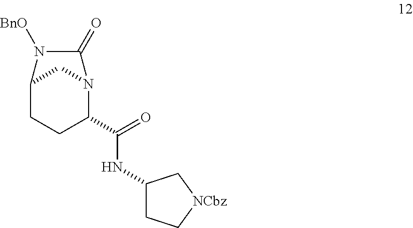

(S)-3-[((2S,5R)-5-Benzyloxyamino-piperidine-2-carbonyl)-amino]-pyrrolidine-1-carboxylic acid benzyl ester di-p-toluoyl-L-tartaric acid salt (54.5 g, 65.0 mmol) was stirred in DCM (540 ml) and 5 wt % sodium bicarbonate (327 ml, 195 mmol) in a 2 L bottle. After stirring for 20 minutes, the mixture was transferred to a 2 L separatory funnel. The organic was washed with water (115 mL), but this failed to remove the residual tartaric acid. The organic layer was washed with 2.5% NaHCO₃ (200 mL), successfully removing the tartaric acid. Assay of the organic layer showed 29.8 g of (S)-3-[((2S,5R)-5-Benzyloxyamino-piperidine-2-carbonyl)-amino]-pyrrolidine-1-carboxylic acid benzyl ester. The organic layer was dried over Na₂SO₄, filtered and evaporated. The residue was dissolved in DCM (510 ml) in a 2-L 3-neck RBF under nitrogen. Hunig's base (38.6 ml, 221 mmol) was added, and the reaction mixture was cooled in an ice-salt bath. Triphosgene (16.39 g, 55.2 mmol) was added portion-wise over 20 minutes at T=−9.1-−5.9° C. The reaction progress was assayed after stirring for an additional 20 minutes, providing a typical LC for this time point. 10% H3PO₄ (300 mL) was added, the bath removed and the reaction mixture allowed to warm to room temperature overnight. At this time, HPLC analysis showed the reaction to be complete. The biphasic mixture was transferred to a 2 L separatory funnel, and the layers were separated. The organic layer was washed with 5% NaHCO₃ (150 mL, pH 8) and water (75 mL). The organic layer was dried over Na2SO4, filtered and solvent-switched to ethanol. Ethanol (60 mL-2 vol) and Heptane (60 mL-2 vol) were added, and the mixture was stirred; crystallization occurred. Additional heptane (240 mL, 8 vol) was added by addition funnel. After stirring for 45 minutes, supernatant assay was 3.74 g (11.1%, based on 33.6 g yield). Additional heptane (60 mL-2 vol, 6:1 total) was added. Supernatant assay showed 3.38 g (10.0%). The suspension was filtered and the solid washed with 9:1 heptane:EtOH, then dried under vacuum with a nitrogen stream, to provide (S)-3-[((2S,5R)-6-benzyloxy-7-oxo-1,6-diaza-bicyclo[3.2.1]octane-2-carbonyl)-amino]-pyrrolidine-1-carboxylic acid benzyl ester (28.2 g, 58.9 mmol, 91% yield) as a white crystalline solid. ¹H NMR (400 MHz, CDCl₃); δ7.32-7.48 (m, 10 H), 6.86 (d, 1H J=7.2 Hz), 5.16 (s, 2H), 5.08 (d, 1H, J=11.4 Hz), 4.95 (d, 1H, J=11.4 Hz), 4.50 (s, 1H, J=5.8 Hz), 3.95 (s, 1H), 3.72 (dd, 1H, J=6.7, 11.4 Hz), 3.52 (s, 2H), 3.25-3.48 (m, 2H), 3.06 (d, 1H, J=11.2 Hz), 2.69 (d, 1H, J=10.4 Hz), 2.40 (dd, 1H, J 6.8, 14.2 Hz), 2.20 (s, 1H), 1.83-2.10 (m, 3H), 1.65 (m, 1H).

### Step 13: (S)-3-[((2S,5R)-6-Hydroxy-7-oxo-1,6-diaza-bicyclo[3.2.1]octane-2-carbonyl)-amino]-pyrrolidine-1-carboxylic acid tert-butyl ester (13)

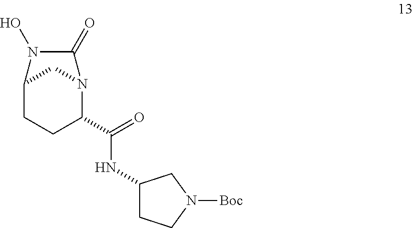

Charged (S)-3-[((2S,5R)-6-Benzyloxy-7-oxo-1,6-diaza-bicyclo[3.2.1]octane-2-carbonyl)-amino]-pyrrolidine-1-carboxylic acid benzyl ester (28.0 g, 58.5 mmol), tetrahydrofuran (392 mL, 4784 mmol), Boc₂O (12.91 mL, 55.6 mmol) and 20 wt % Pd/C (21 g, 29.9 mmol) to a 1-L autoclave. The suspension was put under hydrogen atmosphere at 45 psi at 25° C. for 5 hours At this time, HPLC shows the reaction to be complete. The suspension was filtered through Solka floc, washing with THF. The solution was concentrated and solvent switched to EtOAc (4 vol-83 mL). Crystallization began at this time. Heptane (165 mL, 8 vol) was added via addition funnel and the suspension aged for 1 hour. The solid was filtered and washed with 3:1 Heptane:EtOAc, then dried under vacuum and nitrogen overnight, providing (S)-3-[((2S,5R)-6-Hydroxy-7-oxo-1,6-diaza-bicyclo[3.2.1]octane-2-carbonyl)-amino]-pyrrolidine-1-carboxylic acid tert-butyl ester (15.08 g, 42.6 mmol, 72.7% yield) as a white crystalline solid. ¹H NMR (400 MHz, CDCl₃): δ 7.40 (s, 1H), 7.10 (s, 1H), 4.51 (m, 1H), 4.04 (s, 1H), 3.81 (s, 1H), 3.60 (s, 1H), 3.46 (s, 3H), 3.26 (s, 1H), 2.99 (s, 1H), 2.39 (s, 1H), 2.10-2.23 (m, 2H), 1.80-2.08 (m, 3 μl), 1.49 (s, 9H).

### Step 14: Sulfuric acid mono-[(2S,5R)-7-oxo-2-((S)-pyrrolidin-3-ylcarbamoyl)-1,6-diaza-bicyclo[3.2.1]oct-6-yl]ester (14)

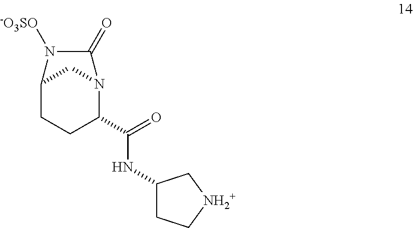

To a 250-mL RBF under nitrogen atmosphere was charged (S)-3-[((2S,5R)-6-Hydroxy-7-oxo-1,6-diaza-bicyclo[3.2.1]octane-2-carbonyl)-amino]-pyrrolidine-1-carboxylic acid tert-butyl ester (8.62 g, 24.32 mmol) and tetrahydrofuran (86 mL, 1050 mmol). 2-picoline (4.82 mL, 48.6 mmol) was added, followed by sulfur trioxide-pyridine complex (13.55 g, 85 mmol). The resulting suspension was stirred for 12 hours. At this time, HPLC shows full conversion. Volatiles were removed under vacuum (65 mL). DCM (100 mL) and water (100 mL) were added to the slurry, followed by dibasic potassium phosphate (6.57 g, 37.7 mmol) and tetrabutylammonium hydrogen sulfate (8.88 g, 26.1 mmol). After stirring for 30 minutes, the biphasic mixture was transferred to a separatory funnel, rinsing/diluting with additional DCM (30 mL). The layers were separated, with nearly all of the pyridine/picoline being washed into the aqueous layer (pH 3-3.5). The organic layer was washed w/22 mL H₂O (pH 4.5), dried over Na₂SO₄, filtered and evaporated. The oil was flushed once with DCM and twice with TFE, then the crude sulfate (16.44 g, 24.32 mmol) was dissolved in TFE (115 ml, 24.32 mmol) in a 500-mL RBF under nitrogen. The solution was cooled to <10° C. in an ice bath. Tetrafluoroboric acid (3.35 ml, 24.32 mmol) was added by syringe and the solution warmed to rt. Bubbles were observed and white solid formed. The resulting suspension was stirred for 18 hours. At this time, HPLC analysis shows nearly 100% conversion to product. Sodium bicarbonate (0.817 g, 9.73 mmol) in water (32.9 ml, 1826 mmol) was added to the slurry and stirred for 5 minutes; all the solid went into solution. Solvent was removed under vacuum (120 mL), leading to formation of a seed bed. 2-Propanol (120 ml, 1558 mmol) was added via addition funnel, and the resulting suspension was stirred for 1 hour, filtered and rinsed with minimal 4:1 2-propanol:water, providing sulfuric acid mono-[(2S,5R)-7-oxo-2-((S)-pyrrolidin-3-ylcarbamoyl)-1,6-diaza-bicyclo[3.2.1]oct-6-yl]ester (4.37 g., 24.3 mmol) as a white crystalline solid. ¹H NMR (DMSO-d₆): δ 8.60 (s, 2H), 8.35 (d, 1H, J=7.0 Hz), 4.38-4.46 (m, 1H), 4.03 (s, 1H), 3.75 (d, 1H, J=6.8 Hz), 3.29-3.35 (m, 3H), 3.17-3.24 (m, 1H), 3.13 (dd, 1H, J=4.7, 11.9 Hz), 2.01-3.04 (m, 1H), 2.05-2.21 (m, 2H), 1.87-1.95 (m, 2H), 1.64-1.74 (m, 2H).

While the foregoing specification teaches the principles of the present invention, with examples provided for the purpose of illustration, the practice of the invention encompasses all of the usual variations, adaptations and/or modifications that come within the scope of the following claims. All publications, patents and patent applications cited herein are incorporated by reference in their entireties into the disclosure, wherein in the case of any inconsistencies, the present disclosure will prevail.

What is claimed is:

1. A process for preparing a compound of Formula III: 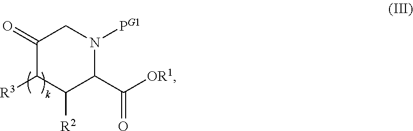
which comprises:
(B) contacting a ketosulfoxonium ylide of Formula II: 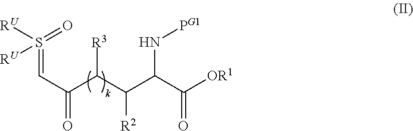
with an iridium catalyst to obtain Compound III; wherein:
P^{G1} is a first amine protective group which forms with the amino nitrogen to which it is attached a carbamate or a benzylamine;
each R^{U} is independently CH₃ or phenyl;
R¹ is C₁₋₆ alkyl or C₁₋₆ alkyl mono- or di-substituted with AryA, wherein each AryA is independently phenyl or napthyl and is optionally substituted with from 1 to 3 substituents each of which is independently halogen, C₁₋₆ alkyl, or O—C₁₋₆ alkyl;
k is an integer equal to 0, 1 or 2; and
R² and R³ are defined as follows:
(a) R² is H, C₁₋₆ alkyl, O—C₁₋₆ alkyl, O—Si(—C₁₋₆ alkyl)₃, or O—Si(—C₁₋₆ lkyl)(-phenyl)₂, and each R³ is H or C₁₋₆ alkyl; or
(b) alternatively and with the proviso that k is 1 or 2, R² and the R³ adjacent to R² together with the carbon atoms to which each is attached form C₅₋₇ cycloalkyl which is optionally substituted with from 1 to 3 substituents each of which is independently C₁₋₆ alkyl, O—C₁₋₆ alkyl, O—Si(—C₁₋₆ alkyl)₃, or O—Si(—C₁₋₆ alkyl)(phenyl)₂; and any other R³ is H or C₁₋₆ alkyl.

2. The process according to claim 1, which further comprises:
(A) contacting a compound of Formula I: 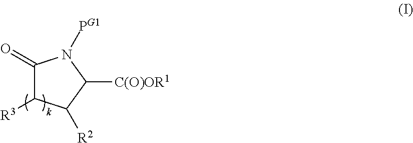
with a sulfoxonium compound of formula (R^{U})₃S(O)Z, wherein at least one R^{U} is CH₃ and Z is halide or tetrafluoroborate, in the presence of strong base to obtain Compound II.

3. The process according to claim 1, which further comprises:
(C) treating Compound III with a reducing agent to obtain a compound of Formula IV: 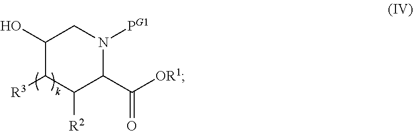
and
(D) contacting Compound IV with a sulfonyl halide of formula IV-Su:
R⁴—SO₂W (IV-Su)
in the presence of a tertiary amine base to obtain a compound of Formula V: 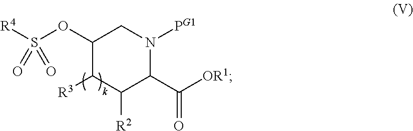
wherein W is halogen; and R⁴ is:
(1) phenyl optionally substituted with from 1 to 3 substituents each of which is independently C₁₋₄ alkyl, C₁₋₄ haloalkyl, O—C₁₋₄ alkyl, O—C₁₋₄ haloalkyl, Cl, Br, F, or NO₂;
(2) C₁₋₄ alkyl; or
(3) C₁₋₄ haloalkyl.

4. The process according to claim 3, which further comprises:
(E) treating Compound V with a P^{G1}-cleaving agent to obtain a compound of Formula VI: 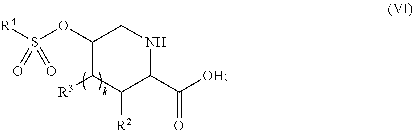
and
(F) treating Compound VI with a P^{G2}-producing agent to obtain a compound of Formula VII: 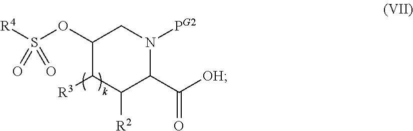
wherein:
P^{G2} is amine protective group which forms with the amino nitrogen to which it is attached an alkyl carbamate.

5. The process according to claim 4, which further comprises:
(G) contacting Compound VII with an azacycloalkylamine of formula VII-Am: 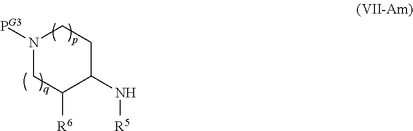
in the presence of a coupling agent to obtain an amide of Formula VIII: 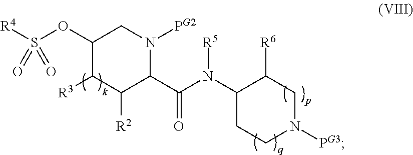
wherein:
P^{G3} is a third amine protective group selected from the group consisting of (i) carbamates other than alkyl carbamates and (ii) benzylamines;
R⁵ is H or C₁₋₃ alkyl;
R⁶ is H, Cl, Br, F, C₁₋₃ alkyl, O—C₁₋₃ alkyl, or N(—C₁₋₃ alkyl)₂;
p is zero, 1 or 2;
q is zero, 1, or 2; and
p+q=zero, 1, 2, or 3.

6. The process according to claim 5, which further comprises:
(H) contacting Compound VIII with N-Boc-O-benzylhydroxylamine in the presence of a base to obtain a compound of Formula IX: 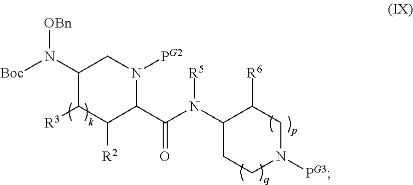
and
(I) treating Compound IX with an acid to obtain a compound of Formula X:

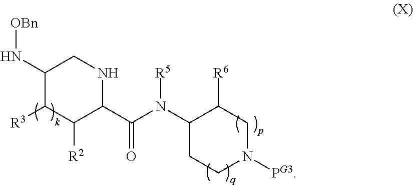

7. The process according to claim 6, which further comprises:
(J) contacting Compound X with phosgene, diphosgene or triphosgene in the presence of a tertiary amine, and then adding an aqueous solution of acid to obtain a compound of Formula XI: 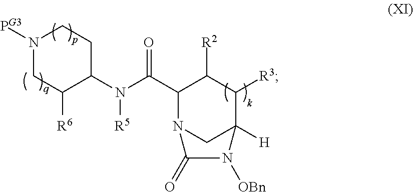
and
(K) contacting Compound XI with a source of hydrogen in the presence of a hydrogenolysis catalyst and in the presence of a Boc-producing agent to obtain a compound of Formula XII:

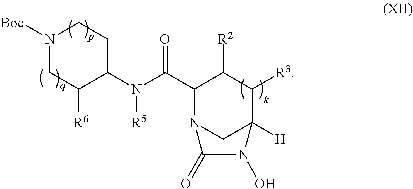

8. The process according to claim 7, which further comprises:
(L) contacting Compound XII with a sulfating agent to obtain a compound of Formula XIII:

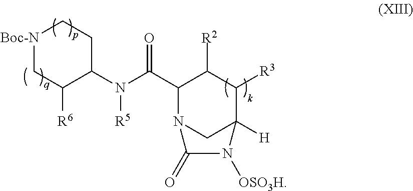

9. The process according to claim 8, which further comprises:
(M) treating Compound XIII with acid to obtain a compound of Formula XIV: 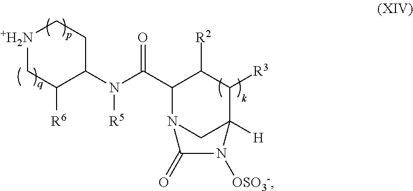
or a salt thereof.

10. A process according to claim 1, wherein the compound of Formula III is Compound 4: 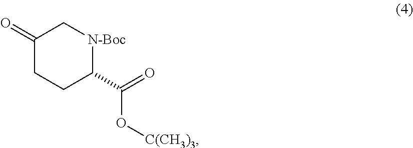
and wherein the process comprises:
(B) contacting ketosulfoxonium ylide 3: 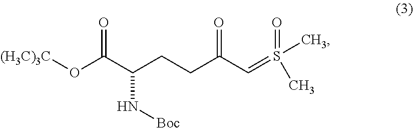
with a catalyst selected from the group consisting of ([Ir(COD)Cl]₂), Ir(COD)₂BF₄, and Ir(COD)₂BARF, to obtain Compound 4.

11. The process according to claim 10, which further comprises:
(A) contacting Compound 2: 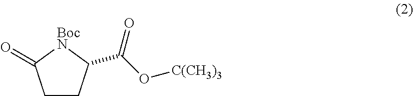
with a trimethylsulfoxonium halide in the presence of a strong base selected from the group consisting of Na C₁₋₄ alkoxides and K C₁₋₄ alkoxides to obtain Compound 3.

12. The process according to claim 10, which further comprises:
(C) treating Compound 4 with a reducing agent selected from the group consisting of Li borohydride, Na borohydride and K borohydride, to obtain Compound 5: 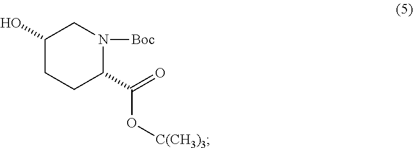
and
(D) contacting Compound 5 with a sulfonyl halide of formula R⁴—SO₂Cl in the presence of a tri-C₁₋₄ alkylamine base to obtain a compound of Formula v: 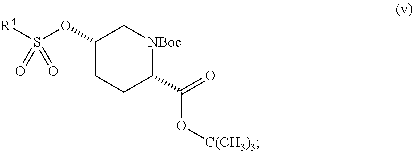
wherein R⁴ is methyl, chloromethyl, phenyl, 4-bromophenyl, 4-trifluoromethylphenyl, 4-methylphenyl, or 2,4-dichlorophenyl.

13. The process according to claim 12, which further comprises:
(E) treating Compound v with acid selected from the group consisting of hydrochloric acid, sulfuric acid, trifluoroacetic acid, and phosphoric acid to obtain Compound vi: 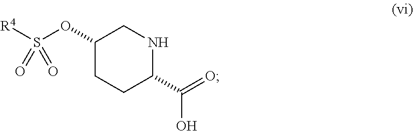
and
(F) treating Compound vi with an Boc-producing agent selected from the group consisting of di-t-butylcarbonate and Sac-ON to obtain a Compound vii:

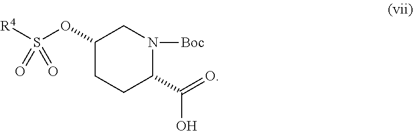

14. The process according to claim 13, which further comprises:
(G) contacting Compound vii with an amine selected from the group consisting of: 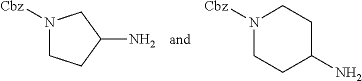
in the presence of a coupling agent to obtain an amide of Formula viii: 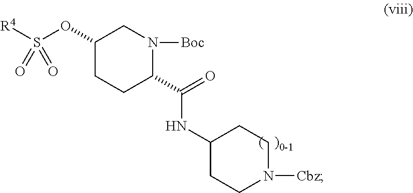
wherein the coupling agent is selected from the group consisting of DCC and EDC.

15. The process according to claim 14, which further comprises:
(H) contacting Compound viii with N-Boc-O-benzylhydroxylamine in the presence of a base selected from the group consisting of K t-butoxide and cesium carbonate to obtain Compound ix: 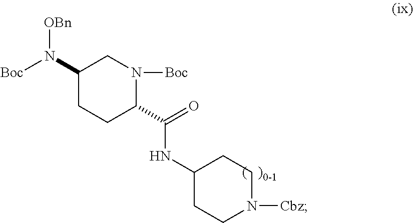
and
(I) treating Compound ix with an acid selected from the group consisting of methanesulfonic acid, chloromethanesulfonic acid, p-toluenesulfonic acid and benzenesulfonic acid to obtain Compound x:

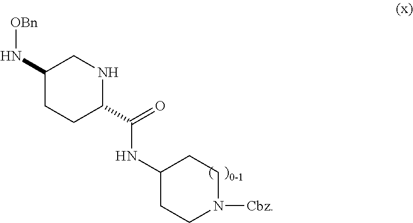

16. The process according to claim 15, which further comprises:
(J) contacting Compound x with triphosgene in the presence of a tri-C₁₋₄ alkylamine base, and then adding an aqueous solution of phosphoric acid to obtain Compound xi: 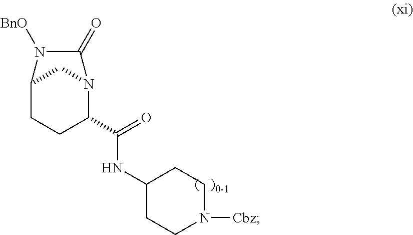
and
(K) contacting Compound xi with hydrogen in the presence of a Pd catalyst and a Bac-producing agent selected from the group consisting of di-t-butylcarbonate and Boc-ON to obtain Compound xii:

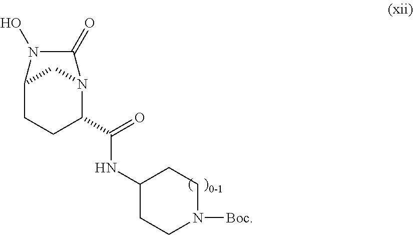

17. The process according to claim 16, which further comprises:
(L) contacting Compound xii with a sulfating agent selected from the group consisting of pyridine-SO₃ complex, chlorosulfonic acid and DMF-SO₃ complex in the presence of 2-picoline to obtain Compound xiii:

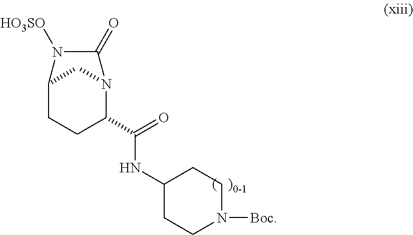

18. The process according to claim 17, which further comprises:
(M) treating Compound xiii with acid to obtain Compound xiv: 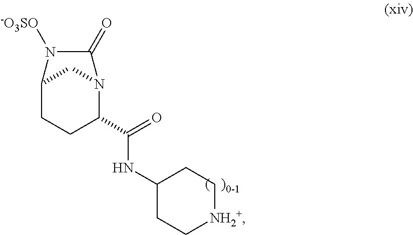
or a salt thereof.

19. A compound selected from the group consisting of: 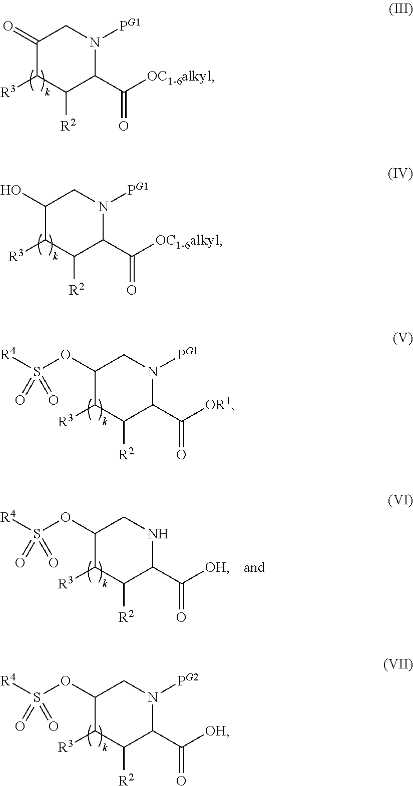
wherein:
P^{G1} is an amine protective group which forms with the amino nitrogen to which it is attached a carbamate or a benzylamine;
P^{G2} is an acid-labile amine protective group which forms with the amino nitrogen to which it is attached a carbamate or a benzylamine;
R¹ is C₁₋₆ alkyl or C₁₋₆ alkyl mono- or di-substituted with AryA, wherein each AryA is independently phenyl or napthyl and is optionally substituted with from 1 to 3 substituents each of which is independently halogen, C₁₋₆ alkyl, or O—C₁₋₆ alkyl;
k is an integer equal to 0, 1 or 2;
R² and R³ are defined as follows:
(a) R² is H, C₁₋₆ alkyl, O—C₁₋₆ alkyl, or Si(—C₁₋₆ alkyl)₃; and each R³ is H or C₁₋₆ alkyl; or
(b) alternatively and with the proviso that k is 1 or 2, R² and the R³ adjacent to R² together with the carbon atoms to which each is attached form C₅₋₇ cycloalkyl which is optionally substituted with from 1 to 3 substituents each of which is independently C₁₋₆ alkyl, O—C₁₋₆ alkyl, O—Si(—C₁₋₆ alkyl)₃, or O—Si(—C₁₋₆ alkyl)(-phenyl)₂; and any other R³ is H or C₁₋₆ alkyl; and
R⁴ is:
(1) phenyl optionally substituted with from 1 to 3 substituents each of which is independently C₁₋₄ alkyl, C₁₋₄ haloalkyl, O—C₁₋₄ alkyl, O—C₁₋₄ haloalkyl, Cl, Br, F, or NO₂;
(2) C₁₋₄ alkyl; or
(3) C₁₋₄ haloalkyl.

20. A compound according to claim 19, which is selected from the group consisting of: 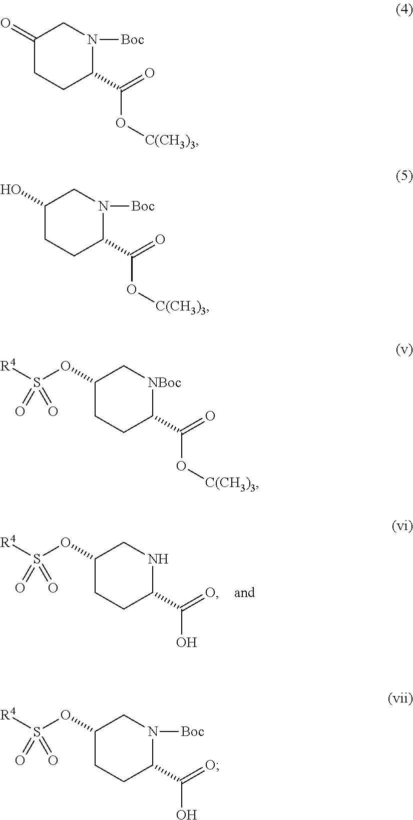
wherein R⁴ is methyl, chloromethyl, phenyl, 4-bromophenyl, 4-trifluoromethylphenyl, 4-methylphenyl, or 2,4-dichlorophenyl.

21. A method for purifying compound 11: 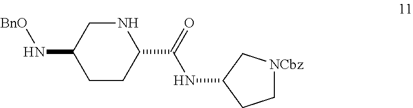
which comprises:
(A) adding an antisolvent to a solution of compound 11 and di-p-toluoyl-L-tartaric acid in an organic solvent to form a suspension of crystals of the di-p-toluoyl-L-tartaric acid salt of 11, and then recovering the crystals; or
(B) adding aqueous hydrochloric acid to a solution of compound 11 in an organic solvent to form a suspension of HCl salt crystals of H, and then recovering the crystals.
